SEQUENCE LISTING (2 sequences)

SEQ ID NO: 1 - DNA, 1530 bp, Variovorax paradoxus
  rRNA (1)..(1530): 16s rRNA gene (rDNA) from Variovorax paradoxus
tggagagtttgatcctggctcagattgaacgctggcggcatgccttacacatgcaagtcgaacggcagcgcgggagcaatcctggcggcgagtggcgaacgggtgagtaatacatcggaacgtgcccaatcgtgggggataacgcagcgaaagctgtgctaataccgcatacgatctacggatgaaagcaggggatcgcaagaccttgcgcgaatggagcggccgatggcagattaggtagttggtgrggtaaaggctcaccaagccttcgatctgtagctggtctgagaggacgaccagccacactgggactgagacacggcccagactcctacgggaggcagcagtggggaattttggacaatgggcgcaagcctgatccagccatgccgcgtgcaggatgaaggccttcgggttgtaaactgcttttgtacggaacgaaacggccttttctaataaagagggctaatgacggtaccgtaagaataagcaccggctaactacgtgccagcagccgcggtaatacgtagggtgcaagcgttaatcggaattactgggcgtaaagcgtgcgcaggcggttatgtaagacagttgtgaaatccccgggctcaacctgggaactgcatctgtgactgcatagctagagtacggtagagggggatggaattccgcgtgtagcagtgaaatgcgtagatatgcggaggaacaccgatggcgaaggcaatcccctggacctgtactgacgctcatgcacgaaagcgtggggagcaaacaggattagataccctggtagtccacgccctaaacgatgtcaactggttgttgggaattcactttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaattcgccagagatggcttagtgctcgaaagagaaccgtaacacaggtgctgcatggctgtcgtcagctcgtgtcgtgagatgttgggttaagtcccgcaacgagcgcaacccttgtcattagttgctacattcagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtcttgtacacaccgcccgtcacaccatgggagcgggttctgccagaagtagttagcttaaccgcaaggagggcgattaccacggcagggttcgtgactggggtgaagtcgtaacaaggtagccgtatcggaaggtgcggytggatcacctcctt

SEQ ID NO: 2 - DNA, 1514 bp, Rhodococcus opacus
  rRNA (1)..(1514): 16s rRNA gene (rDNA) from Rhodococcus opacus
tggagagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagytcgtaggcggtttgtcgcgtcgtctgtgaaaactcrmrgctcaacckygagcytgcaggcgatacgggcagacttgagtactgcaggggagactggaattcctggtgtagcggtgaaatgcgcagatatcaggaggaacaccggtggcgaaggcgggtctctgggcagtaactgacgctgaggarcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccrgtacagagggctgcgakaccgtgaggtggagcgaatcccttaaagcyggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggytggatcacctcctt

(19) United States
(12) Patent Application Publication
(10) Pub. No.: US 2005/0009151 A1
(43) Pub. Date: Jan. 13, 2005

(54) Methods for the stereospecific and enantiomeric enrichment of beta-amino acids

(75) Inventors: Matthew Chase, Grayslake, IL (US); Robert Clayton, Foristell, MO (US); Bryan Landis, Manchester, MO (US); Amit Banerjee, Chesterfield, MO (US)

Correspondence Address: PHARMACIA CORPORATION;GLOBAL PATENT DEPARTMENT, POST OFFICE BOX 1027, ST. LOUIS, MO, 63006, US

(73) Assignee: Pharmacia Corporation

(21) Appl. No.: 10/875,161

(22) Filed: Jun. 22, 2004

Related U.S. Application Data
(60) Provisional application No. 60/486,032, filed on Jul. 10, 2003.
(60) Provisional application No. 60/499,622, filed on Sep. 2, 2003.

(51) Int. Cl. C12P 13/04

(52) U.S. Cl. 435/106

(57) ABSTRACT

The present invention relates to methods for the stereospecific synthesis and for the enantiomeric enrichment of β-amino acids. A novel D-β-aminotransferase, which exhibits stereoselectivity for D-β-phenylalanine, (D-3 amino-3-phenylpropinine acid) was purified from a newly-isolated strain of Variouorax paradoxus. A novel L-β-aminotransferase was purified from a newly-isolated strain of Alcaligenes eutrophus. The D- and L-β-aminotransferases can be used to facilitate the stereoselective biosynthesis of β-D-phenylalanine or β-L-phenylalanine, from a mixture of L-glutamic acid or L-alanine, respectively, and 3-keto-3-phenylpropionic acid in the presence of the cofactor pyridoxal phosphate.

This application claims priority to U.S. Provisional application No. 60/486,032, filed Jul. 10, 2003 and U.S. Provisional application No. 60/499,622, filed Sep. 2, 2003

### FIELD

The present invention relates to methods for the stereospecific synthesis and for the enantiomeric enrichment of β-amino acids.

### BACKGROUND

β-amino acids are often key structural components in a variety of natural products with antibiotic, antifungal, or cytotoxic properties. Synthesis of compounds that require β-amino acids as a component of a final structure, or require use of an enantiomerically-pure β-amino acid as a chiral intermediate, is often challenging (Reviewed in Enantioselective Synthesis of β-Amino Acids, Edited by Eusebio Juaristi, Wiley-VCH, 491 pages, 1997).

Many β-amino acids are not commercially available, are difficult to synthesize, or are prohibitively expensive to obtain as enantiomerically-pure preparations. While many synthetic chemical routes have been developed to produce enantiomerically-pure β-amino acids, biosynthetic routes for producing β-amino acids using aminotransferases or other enzymes may be preferred over chemical methods that require resolution of chiral compounds by chromatographic methods (Soloshonok, V. A. Biocatalytic entry to enantiomerically pure β-amino acids. In Enantioselective Synthesis of β-Amino Acids, Edited by Eusebio Juaristi, Wiley-VCH, 1997, 443-464).

Aminotransferases (E. C. 2.6.1) catalyze the transfer of an amino group, a pair of electrons, and a proton from a primary amine to the carbonyl group of an acceptor molecule (Stirling, D. I. “Enzymic synthesis and resolution of enantiomerically pure compounds” In Chiralty Ind. (1992) 209-22, Wiley, Ed(s). Colins, Andrew N.; Sheldrake, G. N., and Crosby, J.). Most aminotransferases require a cofactor, the coenzyme pyridoxal-5-phosphate. A variety of aminotransferases have been characterized, particularly those involved in the transfer of an amino group from alpha amino acids to 2-keto acids. Pyruvic acid, oxaloacetate, and 2-ketoglutaric acid are important substrates for these enzymes, classified as alpha aminotransferases.

Several other types of aminotransferases (including γ-, ε-, and ω-amiriotransferases) have been described. The ω-aminotransferases can utilize substrates where the amino group is not vicinal (adjacent) to a carboxylate group. In this class of enzymes, the amino donor is generally restricted to ω-amino acids and α,ω-diamino acids. These include enzymes that preferentially use alpha omega-diamino acid (E.C.2.6.8), L-ornithine (E.C.2.6.13), β-alanine (E.C.2.6.18), 4 aminobutyrate (E.C.2.6.19), alpha omega diamine (E.C.2.6.29), L-lysine (E.C.2.6.36), 2,4 diaminobutyrate (E.C.2.6.46), or taurine (E.C.2.6.55) as amino donors and use either 2-ketoglutarate or pyruvate as amino acceptors.

The ω-aminotransferase commonly known as β-alanine-pyruvate transaminase (EC 2.6.1.18; β-alanine-pyruvate aminotransferase; β-alanine-α-alanine transaminase; L-alanine:3-oxopropanoate aminotransferase) carries out the reaction
L-alanine+3-oxopropanoate=pyruvate+β-alanine
using pyridoxal-phosphate as a cofactor (Hayaishi, O., Nishizuka, Y., Tatibana, M., Takeshita, M. and Kuno, S. Enzymatic studies on the metabolism of β-alanine. J. Biol. Chem. 236 (1961) 781-790; Stinson, R. A. and Spencer, M. S. β-Alanine aminotransferase(s) from a plant source. Biochem. Biophys. Res. Commun. 34 (1969) 120-127). Waters and coworkers have noted that the complete catabolism of β-alanine by Pseudomonas aeruginosa involved the transamination with β-alanine:pyruvate aminotransferase (FEMS Micro Lett 34 (1986) 279-282).

Yonaha and coworkers described an omega amino acid pyruvate transaminase found in a Pseudomonas species for which pyruvate was the exclusive amino acceptor (Agric. Biol. Chem. 42(12): 2363-2367, 1978; Agric. Biol. Chem. 41(9): 1701-1706, 1977). Primary aminoalkanes were the preferred amino donors and omega amino acids, such as β-alanine, were not preferred substrates.

Nakano and coworkers identified two omega amino acid transaminases in Bacillus cereus, including a α-alanine aminotransferase and a gamma aminobutyrate transaminase. The two enzymes differed in their activities on β-alanine (100 vs. 3) and gamma aminobutyrate (43 vs. 100) (J. Biochem 81, 1375-1381, 1977).

The β-alanine aminotransferases use β-alanine (3-aminopropionic acid) or straight chain amino acids of similar structure in which the amino group is terminal, and not vicinal, to the carboxylic acid. None of these enzymes, however, have been shown to catalyze the reversible transamination of more complex β-amino acids (e.g., β-substituted β-amino acids, or α,β-di-substituted β-amino acids) such as the transamination between 3-keto-3-phenylpropionic acid and β-phenylalanine.

E. coli penicillin acylase (PA; EC 3.5.1.11) has been used to prepare β-substituted and α,β-di-substituted β-amino acids. Unlike many amino acid acylases and aminotransferases, which tolerate only α-amino acids as substrates, PA appears to have a broad substrate tolerability (Pohl, T., Waldmann H, Tetrahedron Lett (1995) 36: 2963-2966; Waldman H. Tetrahedron Lett (1988) 29:1131-1134). Soloshonok and coworkers have reported the PA-catalyzed hydrolysis of N-phenylacetyl derivatives of β-alkyl and β-fluoroalkyl beta alanines, and the synthesis of optically pure β-aryl-β-amino acids (Soloshonok, V. A. et al., An enzymatic entry to enantiopure β-amino acids. Synlett (1933) 5: 339-341; Soloshonok, V. A. et al., Biocatalytic approach to enantiomerically pure β-amino acids. Tetrahedron: Asymmetry (1995) δ: 1601-1610). Cardillo and coworkers have prepared a series of racemic α-alkyl-β-amino acids (Cardillo G., et al., Enzymic resolution of α-alkyl β-amino acids using immobilized Penicillin G acylase, J. Org. Chem. (1996) 61: 8651-8654). Ng and coworkers have reported the PA-catalyzed resolution of β-monosubstituted beta amino acids (reviewed in Ng, J. S. and Topgi, R. S. Biocatalytic process for optically active β-amino acids. Curr. Opin. Drug Disc Dev (1998) 1(3):314-328; U.S. Pat. No. 6,214,909).

The use of selective enrichment for isolation of aminotransferase enzymes for use in chiral amino acid and amine production has been reported (Stirling, D. I., 1992. The Use of Aminotransferases for the Production of Chiral Amino Acids and Amines, pp. 209-222. In: Collins, Sheldrake, and Crosby (eds), Chirality in Industry, John Wiley and Sons Ltd., New York). Stirling describes the enrichment of microorganisms by including secondary amines in the culture medium as the sole source of nitrogen. Isolated organisms, as well as organisms known to contain ω-aminotransferase activities, were screened for the ability to deaminate 1-phenyl-3-aminobutane. Bacillus megaterium, Pseudomonas aeruginosa ATCC 15692 and Pseudomonas putida ATCC 39213 were all selected for this ability. All of these enzymes were found to require pyruvic acid, or an alternative α-keto acid, as an amino group acceptor in the deamination reaction. While this work describes the accessibility of β-phenylalanine for deamination (an enantiomeric enrichment, or resolution), it shows no evidence of enantioselective synthesis of this compound from the corresponding β-keto acid using a transaminase enzyme. The resolution of racemic β-phenylalanine is also specific for activity on the (R) or L-enantiomer.

U.S. Pat. Nos. 4,950,606, 5,300,437, and 5,169,780 to Stirling et al., disclose the enantiomeric enrichment of amines in which the amino group is on a secondary carbon atom that is chirally-substituted. The stereoselective synthesis of one chiral amine from prochiral ketones are disclosed. The source of the (ω-transaminases used in these studies includes Bacillus megaterium, Pseudomonas aeruginosa ATCC 15692 and Pseudomonas putida ATCC 39213. Production of β-amino acids, such as β-phenylalanine, from β-keto acids using these enzymes was not disclosed.

U.S. Pat. No. 5,316,943 to Kidman describes a method for the production of an optically pure L-amino acid from a D,L racemic mixture of the amino acid comprising the steps of: (i) treating the racemic mixture of the amino acid with a transaminase-producing microorganism; (ii) fermenting said racemic mixture of the amino acid and microorganism at a suitable temperature and pH for a suitable period of time; and (iii) recovering said optically pure L-amino acid.

U.S. Pat. No. 4,518,692 to Rozzell discloses a process for producing alpha amino acids or derivatives thereof, comprising reacting an alpha-keto acid with L-aspartic acid in the presence of transaminase enzyme to produce (1) an alpha amino acid corresponding to said alpha keto acid and (2) oxaloacetate; and decarboxylating said oxaloacetate.

U.S. Pat. No. 4,826,766 to Rozzell discloses a process for producing a desired alpha-amino acid, using a coupled reaction involving two aminotransferases. A first transaminase efficiently catalyzes a reaction resulting in the desired alpha-amino acid and an undesired alpha keto acid, and a second transaminase efficiently catalyzes a reaction using the undesired alpha keto acid as substrate.

U.S. Pat. No. 6,197,558 to Fotheringham, describes a process for making an amino acid by reacting a first amino acid and a keto acid with a transaminase to produce a second amino acid and pyruvate, and reacting the pyruvate with acetolactate synthase to produce a compound that does not react with the transaminase.

U.S. Pat. No. 4,600,692 to Wood, describes a method for the preparation of phenylalanine which comprises contacting phenylpyruvic acid or phenylpyruvate with immobilized whole cells having transaminase activity in the presence of an amine donor. Ruptured or permeabilized cells with the enzyme in the free or immobilized state may also be used.

### SUMMARY

The present invention relates to methods for the stereospecific synthesis and for the enantiomeric enrichment of β-amino acids. In its broadest sense, the present invention involves the use of a β-amino acid transaminase (β-transaminase or β-aminotransferase) in the presence of an amino acceptor to stereoselectively synthesize or enantiomerically enrich a mixture of chiral amines in which the amino group is bound to a non-terminal, chirally-substituted, carbon atom.

One aspect of the invention is a process for the stereoselective synthesis of a β-amino acid, or a salt thereof, the process comprising contacting an amino donor and an amino acceptor in the presence of a β-amino acid transaminase to form a β-amino acid enantiomer, or a salt thereof, from the amino acceptor. In a preferred aspect the β-amino acid is a compound of Formula I 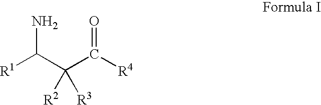
and the amino acceptor is a compound of Formula II 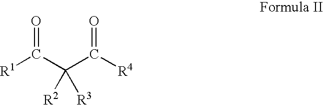
wherein R¹, R², and R³ are independently selected from the group consisting of hydrogen, C₁₋₈ alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl;
wherein R¹, R², and R³ are not all H; and
R⁴ comprises hydroxy, O⁻, and —OM; wherein M is a cation.

Another aspect of the invention is a process for the stereoselective synthesis of a β-amino acid, or a salt thereof, the process comprising contacting an amino donor and an amino acceptor in the presence of a β-amino acid transaminase to stereoselectively form a β-amino acid enantiomer, or a salt thereof, from the amino acceptor;
wherein the β-amino acid, or a salt thereof, is a compound of Formula III 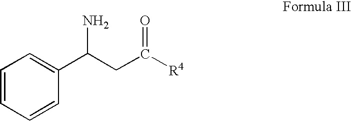
and the amino acceptor is a compound of Formula IV: 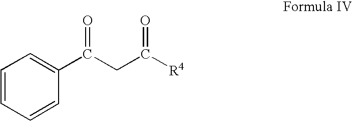
wherein R⁴ comprises hydroxy, O⁻, and —OM; wherein M is a cation.

Another aspect of the invention is a process for enantiomerically enriching a mixture comprising a D-β-amino acid enantiomer and its corresponding L-β-amino acid enantiomer, the process comprising contacting the L-β-amino acid enantiomer with an amino acceptor in the presence of a stereoselective L-β-transaminase to convert at least a portion of the L-β-amino acid enantiomer to the corresponding β-keto acid thereby increasing the molar ratio of the D-β-amino acid enantiomer to the L-β-amino acid enantiomer in the enriched mixture.

Another aspect of the invention is a process for enantiomerically enriching a mixture comprising an L-β-amino acid enantiomer and its corresponding D-β-amino acid enantiomer, the process comprising contacting the D-β-amino acid enantiomer with an amino acceptor in the presence of a stereoselective D-β-transaminase to convert at least a portion of the D-β-amino acid enantiomer to the corresponding β-keto acid thereby increasing the molar ratio of the L-β-amino acid enantiomer to the D-β-amino acid enantiomer in the enriched mixture.

Another aspect of the invention is a method for preparing an enantiomerically enriched β-amino acid, or a salt thereof, which comprises contacting
(i) a racemic β-amino acid, or salt thereof, having the structure of Formula I: 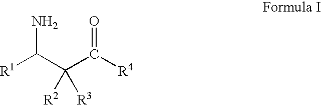
wherein R¹, R², and R³ are independently selected from the group consisting of hydrogen, C₁₋₈ alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl;
wherein R¹, R², and R³ are not all H; and R⁴ comprises hydroxy, O—, and —OM; wherein M is a cation;
(ii) an amino acceptor, and
(iii) a stereospecific β-amino acid transaminase;
under conditions appropriate to convert one enantiomer of the racemic β-amino acid to its corresponding β-keto acid derivative, whereby the opposite enantiomer of the β-amino acid is retained in substantially enantiomerically enriched form, and separating the β-keto acid derivative from the retained β-amino acid.

Another aspect of the invention is a purified stereoselective D-β-transaminase derived from a microorganism selected from the group consisting of Variovorax, Nocardia, Comamonas, Rhodococcus, and Pseudomonas.

Another aspect of the invention is a purified stereoselective L-β-transaminase derived from a microorganism of the genus Alcaligenes.

Another aspect of the invention is a process for purifying a stereospecific β-transaminase from a cell homogenate comprising the stereospecific β-transaminase, the process comprising contacting the cell homogenate with a precipitating agent to yield a precipitate comprising the stereospecific β-transaminase.

Another aspect of the invention is a process for purifying a stereospecific β-transaminase from a composition comprising a stereospecific β-transaminase, the process comprising the steps of: (a) adsorbing the stereospecific β-transaminase onto an hydrophobic interaction material, and (b) eluting the stereospecific β-transaminase from the hydrophobic interaction material using an elution buffer.

Another aspect of the invention is a process for purifying a stereospecific β-transaminase from a composition comprising a stereospecific β-transaminase, the process comprising the steps of: (a) adsorbing the stereospecific β-transaminase onto a size exclusion material, and (b) eluting the stereospecific β-transaminase from the size exclusion material using an elution buffer.

Another aspect of the invention is a process for enriching a population of microorganisms for one or more microorganisms expressing a β-transaminase, the process comprising growing the population of microorganisms in a culture medium comprising a β-amino acid, or a salt thereof, as a selective nitrogen source.

Another aspect of the invention is a purified culture comprising Variovorax paradoxus, wherein the sequence of the 16S rDNA of said Variovorax paradoxus comprises SEQ ID NO: 1.

Another aspect of the invention is a purified culture comprising Rhodococcus opacus, wherein the sequence of the 16S rDNA of said Rhodococcus opacus comprises SEQ ID NO: 2.

Another aspect of the invention is a purified nucleic acid comprising the 16S rDNA sequence set forth in SEQ ID NO: 1, or its complement.

Another aspect of the invention is a nucleic acid that specifically hybridizes under high stringency conditions to a purified nucleic acid comprising the 16S rDNA sequence set forth in SEQ ID NO: 1, or its complement.

Another aspect of the invention is a nucleic acid fragment comprising a fragment of the 16S rDNA sequence set forth in SEQ ID NO: 1, or its complement, having a length of 300 to 1500 nucleotides.

Another aspect of the invention is a nucleic acid that specifically hybridizes under high stringency conditions to nucleic acid fragment comprising a fragment of the 16S rDNA sequence set forth in SEQ ID NO: 1, or its complement, having a length of 300 to 1500 nucleotides.

Another aspect of the invention is a purified nucleic acid comprising the 16S rDNA sequence set forth in SEQ ID NO: 2, or its complement.

Another aspect of the invention is a nucleic acid that specifically hybridizes under high stringency conditions to a purified nucleic acid comprising the 16S rDNA sequence set forth in SEQ ID NO: 2, or its complement.

Another aspect of the invention is a nucleic acid fragment comprising a fragment of the 16S rDNA sequence set forth in SEQ ID NO: 2, or its complement, having a length of 300 to 1500 nucleotides.

Another aspect of the invention is a nucleic acid specifically hybridizes under high stringency conditions to a nucleic acid fragment comprising a fragment of the 16S rDNA sequence set forth in SEQ ID NO: 2, or its complement, having a length of 300 to 1500 nucleotides.

Another aspect of the invention is a method of detecting a nucleic acid comprising: (A) incubating a first nucleic acid with a second nucleic acid obtained or derived from a cell, wherein the first nucleic acid comprises at least 50 nucleotides of SEQ NO: 1, its RNA equivalent, or their full complements, or a nucleic acid with at least 97% identity to about 100 nucleotides of SEQ NO:1, its RNA equivalent, or their full complements, (B) permitting hybridization between said first nucleic acid and said second nucleic acid; and (C) detecting the presence of hybridization to said first nucleic acid.

Another aspect of the invention is a method of detecting a nucleic acid comprising: (A) incubating a first nucleic acid with a second nucleic acid obtained or derived from a cell, wherein the first nucleic acid comprises at least 50 nucleotides of SEQ NO: 2, its RNA equivalent, or their full complements, or a nucleic acid with at least 97% identity to about 100 nucleotides of SEQ NO: 2, its RNA equivalent, or their full complements; (B) permitting hybridization between said first nucleic acid and said second nucleic acid; and (C) detecting the presence of hybridization to said first nucleic acid.

### Terms and Definitions

The following is a list of abbreviations and the corresponding meanings as used interchangeably herein:
CV=column volume(s)
GC/MS=Gas chromatography mass spectrometry
GC-FAME=Gas chromatography fatty acid methyl ester
HPLC=high performance liquid chromatography
L=liter(s)
LC/MS=Liquid chromatography mass spectrometry
mBar=millibar
mg=milligram(s)
ml or mL=milliliter(s)
MWCO=molecular weight cut-off
OD₆₆₀=Optical density in absorbance units
rpm=revolutions per minute
RT=room temperature
U=units
ug or pg=microgram(s)
ul or pl=microliter(s)

The following is a list of one letter abbreviations for various amino acids as used interchangeably herein: A=alanine; B=aspartate or asparagine; C=cysteine; D=aspartate; E=glutamate; F=phenylalanine; G=glycine; H=histidine; I=isoleucine; K=lysine; L=leucine; M=methionine; N=asparagine; P=proline; Q=glutamine; R=arginine; S=serine; T=threonine; U=selenocysteine; V=valine; W=tryptophan; Y=tyrosine; Z=glutamate or glutamine; X=any amino acid residue.

The terms “α-amino acid transaminase”, “β-transaminase”, and “β-amino transferase” are used interchangeably, and mean an enzyme which exhibits the property of reversibly converting the amino group (>C—NH₂) of a β-amino acid, or a salt thereof, to a carbonyl group (>C═O).

The term “beta amino acid” means compounds selected from the group consisting of (1) α-mono-substituted β-amino acids, including β-amino-α-hydroxy acids; (2) α,α-di-substituted β-amino acids; and (3) β-substituted β-amino acids, and salts thereof. The term includes compounds of Formula I 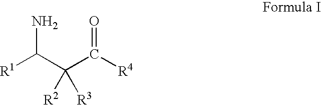
wherein R¹, R², and R³ are independently selected from the group consisting of hydrogen, C₁₋₈ alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals; wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl; wherein R¹, R², and R³ are not all H; and R⁴ comprises hydroxy, O⁻, and —OM; wherein M is a cation.

The term “amino acceptor” means carbonyl compounds which are capable of accepting an amino group from the depicted amine under the influence of a β-amino acid transaminase.

The term “amino donor” refers to various amino compounds which are capable of donating an amino group to the depicted ketone, thereby becoming a carbonyl species under the influence of the same β-amino acid transaminase.

As used herein, alkyl, alkenyl and alkynyl groups, whether as substituents themselves or as portions of substituents, are C₁-C₅₀, with C₁-C₂₀ preferred and C₁-C₁₀ most preferred.

The term “alicyclic hydrocarbon” means an aliphatic radical in a ring with 3 to about 10 carbon atoms, and preferably from 3 to about 6 carbon atoms. Examples of suitable alicyclic radicals include cyclopropyl, cyclopropylenyl, cyclobutyl, cyclopentyl, cyclohexyl, 2-cyclohexen-1-yl, cyclohexenyl, and the like.

The terms “alkyl” and “lower alkyl”, refer to a straight chain or branched chain hydrocarbon radical having 1 to about 10 carbon atoms, and 1 to about 6 carbon atoms, respectively. Examples of such alkyl radicals and lower alkyl radicals are methyl, ethyl, n-propyl, isopropyl, n-butyl, isobutyl, sec-butyl, t-butyl, pentyl, neopentyl, hexyl, isohexyl, octyl, nonyl, decyl, and the like.

The terms “alkenyl” or “lower alkenyl”, refer to unsaturated acyclic hydrocarbon radicals containing at least one double bond and 2 to about 10 carbon atoms, and 2 to about 6 carbon atoms, respectively, which carbon-carbon double bond may have either cis or trans geometry within the alkenyl moiety, relative to groups substituted on the double bond carbons. Examples of such groups are ethenyl, propenyl, butenyl, isobutenyl, pentenyl, hexenyl, octenyl, nonenyl, decenyl, and the like.

The term “alkoxy” refers to straight or branched chain oxy-containing radicals of the formula —OR, wherein R is an alkyl group as defined above. Examples of alkoxy groups encompassed include methoxy, ethoxy, n-propoxy, n-butoxy, isopropoxy, isobutoxy, sec-butoxy, t-butoxy, octyloxy, nonyloxy, decyloxy, and the like.

The terms “alkynyl” or “lower alkynyl”, refer to acyclic hydrocarbon radicals containing one or more triple bonds and 2 to about 10 carbon atoms, and 2 to about 6 carbon atoms, respectively. Examples of such groups are ethynyl, propynyl, butynyl, pentynyl, hexynyl, octynyl, nonynyl, decynyl, and the like.

The term “aromatic hydrocarbon radical” means 6 to about 14 carbon atoms, preferably 6 to about 12 carbon atoms, more preferably 6 to about 10 carbon atoms. Examples of suitable aromatic hydrocarbon radicals include phenyl, naphthyl, and the like.

The term “aryl” as used herein denotes aromatic ring systems composed of one or more aromatic rings. Preferred aryl groups are those consisting of one, two or three aromatic rings. The term embraces aromatic radicals such as phenyl, pyridyl, naphthyl, thiophene, furan, biphenyl and the like.

The terms “arylalkyl” or “aralkyl” refer to a radical of the formula —R²—R¹ wherein R¹ is aryl as defined herein and R² is an alkylene as defined herein. Examples of aralkyl groups include benzyl, pyridylmethyl, naphthylpropyl, phenethyl and the like.

The term “carboxyl derivatives” includes carboxylic acids, carboxylic esters and carboxylic amides.

The term “cycloalkyl” as used herein means saturated or partially unsaturated cyclic radicals containing 3 to about 8 carbon atoms and more preferably 4 to about 6 carbon atoms. Examples of such cycloalkyl radicals include cyclopropyl, cyclopropenyl, cyclobutyl, cyclopentyl, cyclohexyl, 2-cyclohexen-1-yl, and the like.

The term “fused aryl” refers to an aromatic ring such as the aryl groups defined above fused to one or more phenyl rings. “Fused aryl” substituents include, but are not limited to, pentalene, indene, naphthalene, azulene, heptalene, biphenylene, asymm-indacene, symm-indacene, acenaphthylene, flourene, phenalene, phenanthrene, anthracene, flouranthene, acephenanthrylene, aceanthrylene, triphenylene, pyrene, chrysene, naphthacene, plejadene, picene, perylene, pentaphene, pentacene, tetraphenylene, hexaphene, hexacene, rubicene, coronene, trinaphthylene, heptaphene, heptacene, pyranthrene, ovalene, indane, acenaphthene, cholanthrene, aceanthrene, acephenanthrene, violanthrene, isovialanthrene.

The term “fused monocyclic heterocycle” refers to a monocyclic heterocycle as defined above with a benzene fused thereto. As used herein “fused monocyclic heterocycle” substituents include but are not limited to benzofuran, benzopyran, benzodioxole, benzothiazole, benzothiophene, benzimidazole pyrrolizine, indolizine, isoindole, 3H-indole, indole, 1H-indazole, purine, 4H-quinolizine, isoquinoline, quinoline, phthalazine, 1,8-naphthyridine, quinoxaline, quinazoline, quinoline, pteridine, 4aH-carbazole, carbazole, phenanthridine, acridine, perimidine, 1,7-phenanthroline, phenazine, phenomercazine, phenarsazine, isothiazole, phenophosphazine, phenotellurazine, phenoselenazine, phenothiazine, isoxazole, furazane, phenoxazine, isochromane, chromane, pyrrolidine, pyrroline, imidazolidine, phenomercurine, isoarsindole, arsindole, isoarsinoline, arsinoline, arsanthridine, arcidarsine, arsanthrene, isophosphindole, phosphindole, isophosphinoline, phosphinoline, phosphanthrene, selenanthrene, benzo[b]thiophene, naphthol[2,3-b]thiophene, thianthrene, phenothiarsine, isobenzofurane, 2H-chromene, xanthene, phenoxantimonine, phenoxarsine, phenoxaphosphine, phenoxatellurine, phenoxaselenin, and phenoxathiine.

The term “halo” means fluoro, chloro, bromo, or iodo.

The term “halogen” means fluorine, chlorine, bromine, or iodine.

The term “haloalkyl” refers to alkyl groups as defined above substituted with one or more of the same or different halo groups at one or more carbon atom. Examples of haloalkyl groups include trifluoromethyl, dichloroethyl, fluoropropyl and the like.

The term “heteroatom” shall mean atoms other than carbon and hydrogen.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “heterocyclyl radical” means a heterocyclyl hydrocarbon radical preferably an aromatic heterocyclyl hydrocarbon radical with 4 to about 10 carbon atoms, preferably about 5 to about 6; wherein 1 to about 3 carbon atoms are replaced by nitrogen, oxygen or sulfur. The “heterocyclyl radical” may be fused to a aromatic hydrocarbon radical or to another heterocyclyl radical. The “heterocyclyl radical” may be saturated, partially saturated, or fully unsaturated. Suitable examples include pyrrolyl, pyridinyl, pyrazolyl, triazolyl, pyrimidinyl, pyridazinyl, oxazolyl, thiazolyl, imidazolyl, indolyl, thiophenyl, furanyl, tetrazolyl, 2-pyrrolinyl, 3-pyrrolinyl, pyrrolidinyl, 1,3-dioxolanyl, 2-imidazolinyl, imidazolidinyl, 2-pyrazolinyl, pyrazolidinyl, isoxazolyn, isothiazolyn, 1,2,3-oxadiazolyn, 1,2,3-triazolyn, 1,3,4-thiadiazolyn, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyn, quinolinyl, and the like.

The term “lower alkyl”, alone or in combination, means an acyclic alkyl radical containing from 1 to about 10, preferably from 1 to about 8 carbon atoms and more preferably 1 to about 6 carbon atoms. Examples of such radicals include methyl, ethyl, n-propyl, isopropyl, n-butyl, isobutyl, sec-butyl, tert-butyl, pentyl, iso-amyl, hexyl, octyl and the like.

The term “lower alkenyl” refers to an unsaturated acyclic hydrocarbon radical in so much as it contains at least one double bond. Such radicals containing from about 2 to about 10 carbon atoms, preferably from about 2 to about 8 carbon atoms and more preferably 2 to about 6 carbon atoms. Examples of suitable alkenyl radicals include propylenyl, buten-1-yl, isobutenyl, penten-1-yl, 2-2-methylbuten-1-yl, 3-methylbuten-1-yl, hexen-1-yl, hepten-1-yl, and octen-1-yl, and the like.

The term “lower alkylene” or “alkylene” as used herein refers to divalent linear or branched saturated hydrocarbon radicals of 1 to about 6 carbon atoms.

The term “lower alkoxy”, alone or in combination, means an alkyl ether radical wherein the term alkyl is as defined above and most preferably containing 1 to about 4 carbon atoms. Examples of suitable alkyl ether radicals include methoxy, ethoxy, n-propoxy, isopropoxy, n-butoxy, iso-butoxy, sec-butoxy, tert-butoxy and the like.

The term “lower alkynyl” refers to an unsaturated acyclic hydrocarbon radicals in so much as it contains one or more triple bonds, such radicals containing about 2 to about 10 carbon atoms, preferably having from about 2 to about 8 carbon atoms and more preferably having 2 to about 6 carbon atoms. Examples of suitable alkynyl radicals include ethynyl, propynyl, butyn-1-yl, butyn-2-yl, pentyn-1-yl, pentyn-2-yl, 3-methylbutyn-1-yl, hexyn-1-yl, hexyn-2-yl, hexyn-3-yl, 3,3-dimethylbutyn-1-yl radicals and the like.

The terms “monocyclic heterocycle” or “monocyclic heterocyclic” refer to a monocyclic ring containing from 4 to about 12 atoms, and more preferably from 5 to about 10 atoms, wherein 1 to 3 of the atoms are heteroatoms selected from the group consisting of oxygen, nitrogen and sulfur with the understanding that if two or more different heteroatoms are present at least one of the heteroatoms must be nitrogen. The substituents include but are not limited to imidazole, furan, pyridine, oxazole, pyran, triazole, thiophene, pyrazole, thiazole, thiadiazole, pyrazole, pyrazine, pyrimidine, pyridazine, thiophene, tellurophene, selenophene, and pyrrole.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, and ethers.

The compounds as shown in the present invention can exist in various isomeric forms and all such isomeric forms are meant to be included. Tautomeric forms are also included, as well as salts of such isomers and tautomers.

The chemical reactions described in this document are generally disclosed in terms of their broadest application to the preparation of the compounds of this invention. Occasionally, the reactions may not be applicable as described to each compound included within the disclosed scope. The compounds for which this occurs will be readily recognized by those skilled in the art. In all such cases, either the reactions can be successfully performed by conventional modifications known to those skilled in the art, e.g., by appropriate protection of interfering groups, by changing to alternative conventional reagents, by routine modification of reaction conditions, and the like, or other reactions disclosed herein or otherwise conventional, will be applicable to the preparation of the corresponding compounds of this invention. In all preparative methods, all starting materials are known or readily made from known starting materials.

### BRIEF DESCRIPTION OF THE FIGURES

FIG. 1 shows three reaction schemes for the enzymatic deamination of β-phenylalanine involving transaminases, dehydrogenases, and ammonia lyases that could be used to organisms with enhanced ability to synthesize β-amino acids. Using these reaction mechanisms as a guide, conditions for microbial selection by enrichment were developed. The enrichments were based on use of β-phenylalanine as a sole source of nitrogen in liquid culture, permitting microbial species possessing these, or other unknown, enzyme systems to be selectively enriched by growth over those species lacking them.

FIG. 2 shows a reaction scheme for the biocatalytic synthesis of D-β-phenylalanine from 3-keto-3-phenylpropionic acid and glutamate in the presence of a stereospecific D-β-aminotransferase. Aspartic acid is used as an amino donor in a coupled reaction converting α-ketoglutarate to glutamate and oxaloacetate in the presence of asp-oxaloacetate transaminase. Oxaloacetate is converted to pyruvate and CO₂ in the presence of oxaloacetate decarboxylase, minimizing the accumulation of α-ketoglutarate and maximizing the accumulation of D-β-Phenylalanine.

FIG. 3 shows a reaction scheme for the biocatalytic synthesis of L-β-phenylalanine and pyruvate from 3-keto-3-phenylpropionic acid and L-alanine in the presence of a stereospecific L-β-aminotransferase. The pyruvate is converted to CO₂ and acetaldehyde in a coupled reaction, maximizing the accumulation of the L-β-phenylalanine.

FIG. 4 is a fragmentation spectrum obtained by mass spectrometry from an authentic sample of DL-β-phenylalanine, showing the presence of an abundant species with an atomic weight of 166.2 Daltons.

FIG. 5 is a fragmentation spectrum obtained by mass spectrometry from an enzymatically-produced sample of β-phenylalanine. 3-keto-3-phenylpropionic acid, L-glutamate, and pyridoxal phosphate were reacted with a D-β-transaminase preparation until the reaction was completed. β-Phenylalanine was recovered from this enzyme reaction by collection of eluting material from an HPLC-based separation for LC/MS analysis. The most abundant species has an atomic weight of 166.2 Daltons.

FIG. 6 is a fragmentation spectrum obtained by mass spectrometry from an authentic sample of DL-β-phenylalanine. The theoretical mass of the authentic sample matches the theoretical mass of DL-β-phenylalanine.

FIG. 7 is a fragmentation spectrum obtained by mass spectrometry from the enzymatically-produced sample of D-β-phenylalanine as described in FIG. 5. The theoretical mass of the authentic sample matches the theoretical mass of DL-β-phenylalanine.

FIG. 8 illustrates the substrate specificity of the (R)-β-phenylalanine specific transaminase isolated from Alcaligenes eutrophus. A crude cell homogenate was prepared from Alcaligenes eutrophus (strain CP-PyrbPhe-I2) cells grown in minimal salts broth (MSB). The conversion of D- or L-β-phenylalanine from a racemic mixture D,L-β-phenylalanine to the corresponding ketone was measured in the presence of various substrates (pyruvate, pyruvate plus the cofactor pyridoxal phosphate (PLP), alpha keto glutarate, alpha keto glutarate plus pyridoxal phosphate). The amount of D-β-phenylalanine (S-b-Phe) or L-β-phenylalanine (R-b-Phe) consumed in the reaction was measured at 1 hour. The rate of conversion of L-β-phenylalanine (R-b-Phe) is greater than rate of conversion for D-β-phenylalanine (S-b-Phe). Pyridoxal phosphate enhances the reaction rate, and pyruvate is a preferred substrate compared to alpha keto glutarate.

FIG. 9 illustrates the substrate specificity of the (R)-β-phenylalanine specific transaminase isolated from Alcaligenes eutrophus. The crude cell homogenate was prepared from Alcaligenes eutrophus (strain CP-PyrbPhe-I2) cells grown in nutrient broth (NB). The assay conditions are the same as those described in the legend to FIG. 8.

### DETAILED DESCRIPTION OF THE INVENTION

In its broadest sense, the present invention involves the use of a β-amino acid transaminase (β-transaminase or β-aminotransferase) in the presence of an amino acceptor to stereoselectively synthesize or enantiomerically enrich a mixture of chiral amines.

The present invention relates to the surprising discovery and partial characterization of β-aminotransferase enzymes from newly-isolated microorganisms (e.g., from soil and compost samples). These enzymes may offer economically feasible routes for the biosynthetic preparation of enantiomerically-pure β-amino acids, particularly L-β-phenylalanine and D-β-phenylalanine.

Microorganisms possessing β-aminotransferase activity were isolated utilizing an enrichment culturing protocol. The protocol enriched microbes from environmental populations based on the ability to utilize nitrogen supplied exclusively as a β-amino acid. DL-β-phenylalanine, when presented as the sole source of nitrogen, resulted in successful selection of organisms capable of utilizing nitrogen attached to the beta carbon of an amino acid. The selected organisms were used as pure cultures and screened for β-aminotransferase activity, both as whole cell catalysts, and as cell-free homogenate preparations. Microorganisms possessing β-aminotransferase activity catalyzed the reversible transamination between 3-keto-3-phenylpropionic acid and β-phenylalanine.

One enzyme exhibits 100% preference for β-D-phenylalanine, having no activity on the L-enantiomer. This enzyme prefers α-ketoglutarate as amino acceptor, and requires pyridoxal phosphate for catalysis. This enzyme does not act on L-amino acids or α-amino acids, and surprisingly, prefers L-glutamic acid over D-glutamic acid as the amino donor in the transamination of 3-keto-3-phenylpropionic acid to β-D-phenylalanine. Under appropriate conditions, this enzyme may be used for enantioselective biosynthesis of β-amino acids, particularly D-β-amino acids, and preferentially D-β-phenylalanine.

A second β-aminotransferase was also isolated under the same conditions described above and demonstrated to have the opposite stereoselectivity. The second enzyme exhibits 100% preference for β-L-phenylalanine, having no activity on the D-enantiomer. This enzyme prefers pyruvic acid as amino acceptor, and requires pyridoxal phosphate for catalysis. Under appropriate conditions, this enzyme may be used for enantioselective biosynthesis of β-amino acids, particularly L-β-amino acids, and preferentially L-β-phenylalanine.

One aspect of the invention is a process for the stereoselective synthesis of a β-amino acid, or a salt thereof, the process comprising contacting an amino donor and an amino acceptor in the presence of a β-amino acid transaminase to form a β-amino acid enantiomer, or a salt thereof, from the amino acceptor.

Preferably the amino acceptor is a β-keto acid.

Preferably the amino donor is an α-amino acid.

Preferably the molar ratio of the D-β-amino acid or L-β-amino acid formed to the respective L-β-amino acid or D-β-amino acid formed is greater than 1:1 More preferably the molar ratio is greater than 3:1. Even more preferably the molar ratio is greater than 10:1.

Preferably the process further comprises recovering the β-amino acid.

Preferably the contacting is carried out in the presence of whole cells of a microorganism which comprises the β-transaminase.

Preferably the contacting is carried out in the presence of permeabilized cells of a microorganism which comprises the β-transaminase.

Preferably the contacting is carried out in the presence of a cell-free preparation of the β-transaminase.

Preferably the β-transaminase is immobilized on a support.

Preferably the contacting is carried out in aqueous conditions.

Preferably the contacting is carried out in the presence of an organic cosolvent. More preferably the organic cosolvent chosen from the group consisting of alcohols, ketones, ethers, esters, nitriles, and hydrocarbons. More preferably the organic cosolvent chosen from the group consisting of methanol, ethanol, propanol, isopropanol, acetone, diethyl ether, ethyl acetate, tetrahydrofuran, dimethylformamide, acetonitrile, methyl t-butyl ether, di-octyl phthalate, toluene, dialkyl ether, diphenyl ether. Even more preferably the organic cosolvent is present in an amount between 0% and 100% (v/v). Even more preferably the organic cosolvent is present in an amount between 0% and about 30% (v/v). Even more preferably the organic cosolvent is present in an amount of about 5% (v/v). More preferably the organic cosolvent is water miscible. More preferably the organic cosolvent is water immiscible.

Preferably the process further comprises reacting the corresponding keto form of the amino donor, produced by contacting an amino donor and an amino acceptor in the presence of a β-amino acid transaminase, under conditions appropriate to produce a compound that does not react with the β-transaminase. More preferably the keto form of the amino donor is an alpha keto acid. Even more preferably the amino donor is glutamate, and the keto form of the amino donor is α-keto glutarate. Even more preferably the amino donor is glutamate, the keto form of the amino donor is α-keto glutarate, and the reacting is carried out in the presence of asp-oxaloacetate transaminase and oxaloacetate decarboxylase. More preferably the keto form of the amino donor is pyruvic acid. Even more preferably the amino donor is L-alanine, the keto form of the amino donor is pyruvic acid, and the reacting is carried out in the presence of pyruvate decarboxylase.

Preferably the β-amino acid enantiomer is a D-β-amino acid enantiomer. More preferably the β-amino acid enantiomer is a D-β-amino acid and the transaminase is a stereoselective D-β-transaminase. Even more preferably the transaminase is derived from a microorganism selected from the genera consisting of Variovorax, Nocardia, Comamonas, Rhodococcus, and Pseudomonas. Even more preferably the transaminase is derived from a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus. Even more preferably the transaminase is substantially identical to a stereoselective D-β-transaminase produced by a microorganism selected from the genera consisting of Variovorax, Nocardia, Comamonas, Rhodococcus, and Pseudomonas. Even more preferably the transaminase is substantially identical to a stereoselective D-β-transaminase produced by a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus. Even more preferably the transaminase is at least 80% identical to the amino acid sequence of a stereoselective D-β-transaminase produced by a microorganism selected from the genera consisting of Variovorax, Nocardia, Comamonas, Rhodococcus, and Pseudomonas. Even more preferably the transaminase is at least 80% identical to the amino acid sequence of a stereoselective D-β-transaminase produced by a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

Preferably the α-amino acid enantiomer is the L-β-amino acid enantiomer. More preferably an L-β-amino acid is synthesized in the presence of a stereoselective L-β-transaminase. Even more preferably the transaminase is derived from a microorganism of the genus Alcaligenes. Even more preferably the transaminase is produced by Alcaligenes eutrophus. Even more preferably the transaminase is substantially identical to a stereoselective L-β-transaminase produced by a microorganism of the genus Alcaligenes. Even more preferably the transaminase is substantially identical to a stereoselective L-β-transaminase produced by Alcaligenes eutrophus. Even more preferably the transaminase is at least 80% identical to the amino acid sequence of a stereoselective L-β-transaminase produced by a microorganism of the genus Alcaligenes. Even more preferably the transaminase is at least 80% identical to the amino acid sequence of a stereoselective L-β-transaminase produced by Alcaligenes eutrophus.

Preferably the β-amino acid enantiomer is the D-β-amino acid enantiomer synthesized in the presence of a stereoselective D-β-transaminase, wherein said transaminase is derived from a microorganism having at least 97% identity with the 16S rRNA sequence set forth in SEQ ID NO:1.

Preferably the β-amino acid enantiomer is the L-β-amino acid enantiomer synthesized in the presence of a stereoselective L-β-transaminase, wherein said transaminase is derived from a microorganism having at least 97% identity with the 16S rRNA sequence set forth in SEQ ID NO:2.

Preferably the β-amino acid is a compound of Formula I 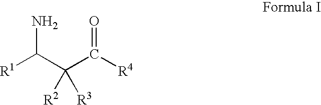
and the amino acceptor is a compound of Formula II 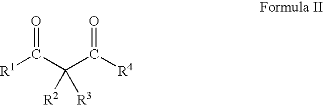
wherein R¹, R², and R³ are independently selected from the group consisting of hydrogen, C₁₋₈ alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl;
wherein R¹, R², and R³ are not all H; and
R⁴ comprises hydroxy, O⁻, and —OM; wherein M is a cation.

More preferably M is selected from the group consisting of alkali metal cations and NH₄ ⁺. Even more preferably M is selected from the group consisting of Na⁺, K⁺, and NH₄ ⁺.

More preferably R¹, R², and R³ are selected from the group consisting of hydrogen, C₁₋₈ alkyl, C₂₋₈ alkenyl, C₂₋₈ alkynyl, C₆₋₁₂ aryl, and C₆₋₁₂ aryl-C₁₋₈ alkyl, radicals; wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl radicals.

Even more preferably R¹, R², and R³ are independently selected from the group consisting of hydrogen, C₆₋₁₂ aryl, and C₆₋₁₂ aryl-C₁₋₈ alkyl, radicals; wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl radicals.

Even more preferably R¹, R², and R³ are selected from the group consisting of hydrogen, C₁₋₈ alkyl, C₂₋₈ alkenyl, and C₂₋₈ alkynyl, radicals; wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl radicals.

Even more preferably R² or R³, but not both, is OH.

Even more preferably R² or R³, but not both, is H.

Even more preferably R² and R³ are both H.

Even more preferably R¹ is selected from the group consisting of C₆₋₁₂ aryl and C₆₋₁₂ aryl-C₁₋₈ alkyl radicals, which are optionally substituted with halogen, nitro, thio, lower alkoxy, and lower alkyl radicals.

Even more preferably R¹ is phenyl.

Another aspect of the invention is a process for the stereoselective synthesis of a β-amino acid, or a salt thereof, the process comprising contacting an amino donor and an amino acceptor in the presence of a β-amino acid transaminase to stereoselectively form a β-amino acid enantiomer, or a salt thereof, from the amino acceptor;
wherein the β-amino acid, or a salt thereof, is a compound of Formula 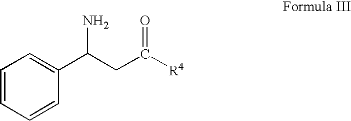
and the amino acceptor is a compound of Formula IV: 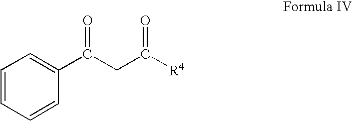
wherein R⁴ comprises hydroxy, O⁻, and —OM; wherein M is a cation.

Preferably the β-amino acid is selected from the group consisting of D-β-phenylalanine and L-β-phenylalanine.

Preferably the amino acceptor is selected from the group consisting of a β-keto acid and a compound converted to β-keto acid in situ.

Preferably the amino donor is selected from the group consisting of: D-glutamic acid, L-glutamic acid, D,L-glutamic acid, D-aspartic acid, L-aspartic acid, D,L-aspartic acid, D-alanine, L-alanine, and D,L-alanine, 3-aminoadipic acid, and 2-aminoadipic acid. More preferably the amino donor is selected from the group consisting of: D-glutamic acid, L-glutamic acid, D,L-glutamic acid, D-aspartic acid, L-aspartic acid, D,L-aspartic acid,

Another aspect of the invention is a process for enantiomerically enriching a mixture comprising a D-β-amino acid enantiomer and its corresponding L-β-amino acid enantiomer, the process comprising contacting the L-β-amino acid enantiomer with an amino acceptor in the presence of a stereoselective L-β-transaminase to convert at least a portion of the L-β-amino acid enantiomer to the corresponding β-keto acid thereby increasing the molar ratio of the D-β-amino acid enantiomer to the L-β-amino acid enantiomer in the enriched mixture.

Preferably the molar ratio of D-β-amino acid enantiomer to L-β-amino acid enantiomer in the enriched mixture is greater than 1:1.

More preferably the molar ratio of D-β-amino acid enantiomer to L-β-amino acid enantiomer in the enriched mixture is greater than 3:1. Even more preferably the molar ratio of D-β-amino acid enantiomer to L-β-amino acid enantiomer in the enriched mixture is greater than 10:1.

Another aspect of the invention is a process for enantiomerically enriching a mixture comprising an L-β-amino acid enantiomer and its corresponding D-β-amino acid enantiomer, the process comprising contacting the D-β-amino acid enantiomer with an amino acceptor in the presence of a stereoselective D-β-transaminase to convert at least a portion of the D-β-amino acid enantiomer to the corresponding β-keto acid thereby increasing the molar ratio of the L-β-amino acid enantiomer to the D-β-amino acid enantiomer in the enriched mixture.

Preferably the molar ratio of L-β-amino acid enantiomer to D-β-amino acid enantiomer in the enriched mixture is greater than 1:1.

More preferably the molar ratio of L-β-amino acid enantiomer to D-β-amino acid enantiomer in the enriched mixture is greater than 3:1. Even more preferably the molar ratio of L-β-amino acid enantiomer to D-β-amino acid enantiomer in the enriched mixture is greater than 10:1.

Another aspect of the invention is a method for preparing an enantiomerically enriched β-amino acid, or a salt thereof, which comprises contacting
(i) a racemic β-amino acid, or salt thereof, having the structure of Formula I: 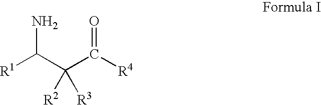
wherein R¹, R², and R³ are independently selected from the group consisting of hydrogen, C₁₋₈ alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl;
wherein R¹, R², and R³ are not all H; and
R⁴ comprises hydroxy, O⁻, and —OM; wherein M is a cation;
(ii) an amino acceptor, and
(iii) a stereospecific β-amino acid transaminase;
under conditions appropriate to convert one enantiomer of the racemic β-amino acid to its corresponding β-keto acid derivative, whereby the opposite enantiomer of the β-amino acid is retained in substantially enantiomerically enriched form, and separating the β-keto acid derivative from the retained β-amino acid.

Another aspect of the invention is a purified stereoselective D-β-transaminase derived from a microorganism selected from the group consisting of Variovorax, Nocardia, Comamonas, Rhodococcus, and Pseudomonas.

Preferably the purified stereoselective D-α-transaminase is derived from a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

More preferably the sequence of the 16S rDNA of said microorganism has at least 97% identity over 1500 nucleotides with the sequence of the 16S rDNA set forth in SEQ ID NO:1. Even more preferably the purified stereoselective D-β-transaminase is derived from Variovorax paradoxus. Even more preferably the purified stereoselective D-β-transaminase is derived from Variovorax paradoxus, wherein the sequence of the 16S rDNA of said microorganism comprises SEQ ID NO: 1. Even more preferably the purified stereoselective D-β-transaminase of claim 83 is derived from Variovorax paradoxus, wherein the sequence of the 16S rDNA of said microorganism consists of SEQ ID NO: 1.

More preferably the sequence of the 16S rDNA of said microorganism has at least 97% identity over 1500 nucleotides with the sequence of the 16S rDNA set forth in SEQ ID NO:2. Even more preferably the purified stereoselective D-β-transaminase is derived from Rhodococcus opacus. Even more preferably the purified stereoselective D-β-transaminase of claim is derived from Rhodococcus opacus, wherein the sequence of the 16S rDNA of said microorganism comprises SEQ ID NO: 2. Even more preferably the purified stereoselective D-β-transaminase is derived from Rhodococcus opacus, wherein the sequence of the 16S rDNA of said microorganism consists of SEQ ID NO: 2.

Another aspect of the invention is a purified stereoselective L-β-transaminase derived from a microorganism of the genus Alcaligenes.

Preferably the purified stereoselective L-β-transaminase is derived from Alcaligenes eutrophus.

Another aspect of the invention is a process for purifying a stereospecific β-transaminase from a cell homogenate comprising the stereospecific β-transaminase, the process comprising contacting the cell homogenate with a precipitating agent to yield a precipitate comprising the stereospecific β-transaminase.

Preferably the precipitating agent is ammonium sulfate.

Preferably the precipitate is further purified by chromatography.

More preferably the precipitate is further purified by hydrophobic interaction chromatography. Even more preferably the hydrophobic interaction chromatography is performed with a butyl sepharose FF resin.

More preferably the precipitate is further purified by size exclusion chromatography. Even more preferably the size exclusion chromatography is performed with a TSK G300 SW resin.

More preferably the precipitate is further purified by hydrophobic interaction chromatography and size exclusion chromatography. Even more preferably the hydrophobic interaction chromatography is performed with a butyl sepharose FF resin and the size exclusion chromatography is carried out with a TSK G300 SW resin.

Preferably the stereoselective D-β-transaminase is produced by the process wherein the cell homogenate is obtained from a microorganism selected from the group consisting of Variovorax, Nocardia, Comamonas, Rhodococcus, and Pseudomonas.

More preferably the stereoselective D-β-transaminase is produced by a the process wherein the cell homogenate is obtained from a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus. Even more preferably the stereoselective D-β-transaminase is produced by the process wherein the cell homogenate is obtained from Variovorax paradoxus. Even more preferably the stereoselective D-β-transaminase is produced by Variovorax paradoxus, wherein the 16S rDNA of said microorganism comprises SEQ ID NO: 1. Even more preferably the stereoselective D-β-transaminase is produced by Variovorax paradoxus, wherein the 16S rDNA of said microorganism consists of SEQ ID NO: 1. Even more preferably the stereoselective D-β-transaminase is produced by the process wherein the cell homogenate is obtained from Rhodococcus opacus. Even more preferably the stereoselective D-β-transaminase is produced by Rhodococcus opacus, wherein the 16S rDNA of said microorganism comprises SEQ ID NO: 2. Even more preferably the stereoselective D-β-transaminase is produced by Rhodococcus opacus, wherein the 16S rDNA of said microorganism consists of SEQ ID NO: 2.

Preferably the stereoselective L-β-transaminase is produced by the process wherein the cell homogenate is obtained from a microorganism of the genus Alcaligenes.

More preferably the stereoselective L-β-transaminase is produced by the process wherein the microorganism is Alcaligenes eutrophus.

Preferably a β-transaminase is produced by the process having a subunit molecular weight between 45 and 55 kDa.

Another aspect of the invention is a process for purifying a stereospecific β-transaminase from a composition comprising a stereospecific β-transaminase, the process comprising the steps of: (a) adsorbing the stereospecific β-transaminase onto an hydrophobic interaction material, and (b) eluting the stereospecific β-transaminase from the hydrophobic interaction material using an elution buffer.

Another aspect of the invention is a process for purifying a stereospecific β-transaminase from a composition comprising a stereospecific β-transaminase, the process comprising the steps of: (a) adsorbing the stereospecific β-transaminase onto a size exclusion material, and (b) eluting the stereospecific β-transaminase from the size exclusion material using an elution buffer.

Another aspect of the invention is a process for enriching a population of microorganisms for one or more microorganisms expressing a β-transaminase, the process comprising growing the population of microorganisms in a culture medium comprising a β-amino acid, or a salt thereof, as a selective nitrogen source.

Preferably, the β-transaminase is a stereospecific β-transaminase.

More preferably the stereospecific β-transaminase is a D-β-transaminase.

More preferably the stereospecific β-transaminase is an L-β-transaminase.

Preferably the D-amino acid is selected from the group consisting of a D-β-amino acid, an L-β-amino acid, or a mixture thereof.

More preferably the β-amino acid is selected from the group consisting of a D-β-phenylalanine, and L-β-phenylalanine, or a mixture thereof.

Preferably the culture medium comprises inorganic salts, a carbon source, and a nitrogen source, wherein said β-amino acid, or a salt thereof, is the nitrogen source used for selective enrichment.

Preferably the culture medium comprises inorganic salts, a carbon source, and a nitrogen source, wherein said β-amino acid, or a salt thereof, is the nitrogen source and the carbon source.

Preferably the population of microorganisms are collected from soil.

Another aspect of the invention is a purified culture comprising Variovorax paradoxus, wherein the sequence of the 16S rDNA of said Variovorax paradoxus comprises SEQ ID NO: 1.

Another aspect of the invention is a purified culture comprising Rhodococcus opacus, wherein the sequence of the 16S rDNA of said Rhodococcus opacus comprises SEQ ID NO: 2.

Another aspect of the invention is a purified nucleic acid comprising the 16S rDNA sequence set forth in SEQ ID NO: 1, or its complement.

Preferably the purified nucleic acid comprises the RNA equivalent of a purified nucleic acid comprising the 16S rDNA sequence set forth in SEQ ID NO: 1, or its complement

Another aspect of the invention is a nucleic acid specifically hybridizes under high stringency conditions to a purified nucleic acid comprising the 16S rDNA sequence set forth in SEQ ID NO: 1, or its complement.

Another aspect of the invention is a nucleic acid fragment comprising a fragment of the 16S rDNA sequence set forth in SEQ ID NO: 1, or its complement, having a length of 300 to 1500 nucleotides.

Preferably the purified nucleic acid comprises the RNA equivalent of a nucleic acid fragment comprising a fragment of the 16S rDNA sequence set forth in SEQ ID NO: 1, or its complement, having a length of 300 to 1500 nucleotides.

Another aspect of the invention is a nucleic acid specifically hybridizes under high stringency conditions to nucleic acid fragment comprising a fragment of the 16S rDNA sequence set forth in SEQ ID NO: 1, or its complement, having a length of 300 to 1500 nucleotides.

Another aspect of the invention is a purified nucleic acid comprising the 16S rDNA sequence set forth in SEQ ID NO: 2, or its complement.

Preferably the purified nucleic acid comprises the RNA equivalent of a purified nucleic acid comprising the 16S rDNA sequence set forth in SEQ ID NO: 2, or its complement.

Another aspect of the invention is a nucleic acid that specifically hybridizes under high stringency conditions to a purified nucleic acid comprising the 16S rDNA sequence set forth in SEQ ID NO: 2, or its complement.

Another aspect of the invention is a nucleic acid fragment comprising a fragment of the 16S rDNA sequence set forth in SEQ ID NO: 2, or its complement, having a length of 300 to 1500 nucleotides.

Preferably the purified nucleic acid comprises the RNA equivalent of a nucleic acid fragment comprising a fragment of the 16S rDNA sequence set forth in SEQ ID NO: 2, or its complement, having a length of 300 to 1500 nucleotides.

Another aspect of the invention is a nucleic acid specifically hybridizes under high stringency conditions to a nucleic acid fragment comprising a fragment of the 16S rDNA sequence set forth in SEQ ID NO: 2, or its complement, having a length of 300 to 1500 nucleotides.

Another aspect of the invention is a method of detecting a nucleic acid comprising: (A) incubating a first nucleic acid with a second nucleic acid obtained or derived from a cell, wherein the first nucleic acid comprises at least 50 nucleotides of SEQ NO: 1, its RNA equivalent, or their full complements, or a nucleic acid with at least 97% identity to about 100 nucleotides of SEQ NO:1, its RNA equivalent, or their full complements, (B) permitting hybridization between said first nucleic acid and said second nucleic acid; and (C) detecting the presence of hybridization to said first nucleic acid.

Preferably said first nucleic acid comprises at least 100 nucleotides of SEQ NO: 1, its RNA equivalent, or their full complements.

Preferably said first nucleic acid comprises at least 150 nucleotides of SEQ NO: 1, its RNA equivalent, or their full complements.

Preferably said first nucleic acid comprises at least 200 nucleotides of SEQ NO: 1, its RNA equivalent, or their full complements.

Preferably said first nucleic acid comprises at least 250 nucleotides of SEQ NO: 1, its RNA equivalent, or their full complements.

Preferably said first nucleic acid comprises at least 300 nucleotides of SEQ NO: 1, its RNA equivalent, or their full complements.

Another aspect of the invention is a method of detecting a nucleic acid comprising: (A) incubating a first nucleic acid with a second nucleic acid obtained or derived from a cell, wherein the first nucleic acid comprises at least 50 nucleotides of SEQ NO: 2, its RNA equivalent, or their full complements, or a nucleic acid with at least 97% identity to about 100 nucleotides of SEQ NO: 2, its RNA equivalent, or their full complements; (B) permitting hybridization between said first nucleic acid and said second nucleic acid; and (C) detecting the presence of hybridization to said first nucleic acid.

Preferably said first nucleic acid comprises at least 100 nucleotides of SEQ NO: 2, its RNA equivalent, or their full complements.

Preferably said first nucleic acid comprises at least 150 nucleotides of SEQ NO: 2, its RNA equivalent, or their full complements.

Preferably said first nucleic acid comprises at least 200 nucleotides of SEQ NO: 2, its RNA equivalent, or their full complements.

Preferably said first nucleic acid comprises at least 250 nucleotides of SEQ NO: 2, its RNA equivalent, or their full complements.

Preferably said first nucleic acid comprises at least 300 nucleotides of SEQ NO: 2, its RNA equivalent, or their full complements.

General Materials and Methods

Racemic β-phenylalanine was from Aldrich Chemical Company. Sodium pyruvate and α-ketoglutarate were purchased from Sigma Chemical Company (St. Louis, Mo.). Enantiomerically pure L- or (R)-3-amino-3-phenylpropionic acid, D- or (S)-3-amino-3-phenylpropionic acid, D- or (R)-3-amino-5-phenyl-pentanoic acid, and L- or (S)-3-amino-5-phenyl-pentanoic acid were purchased from PepTech Corporation (Cambridge, Mass.). D- or (R)-2phenylglycine and L- or (S)-2-phenylglycine were purchased from Sigma. Bacto agars and dehydrated media were purchased from Difco. Bradford Reagent for protein determination and Bovine Serum Albumin Standard Solution were from Sigma. All other reagents were of analytical grade.

All parts are by weight, and temperatures are in degrees centigrade (° C.), unless otherwise indicated.

Microorganisms

Microorganisms characterized in this work were isolated from environmental samples by selective enrichment as described below.

Culture Media

Enrichment cultures were carried out in Slater's Enrichment Medium (Slater et al., 1979). This medium comprised 1.5 g/L of KHPO₄, 0.5 g/L of KH₂PO₄, 0.5 g/L of (NH₄)₂SO₄, 0.2 g/L of MgSO₄·7H₂O₂, and 10.0 ml of Trace solution in Milli-Q™ H₂O (Milli-Q is a trademark of Millipore Corporation for its water purification systems), at pH 7.0. Trace solution comprised 12.0 g/L of Na₂EDTA·2H₂O, 2.0 g/L of FeSO₄·7H₂₀, 1.0 g/L of CaCl₂, 10.0 g/L of Na₂SO₄, 0.4 g/L of ZnSO₄·7H₂O, 0.4 g/L of MnSO₄·4H₂O, 0.1 g/L of CuSO₄·5H₂O, 0.1 g/L of Na₂MoO₄·2H₂O, and 0.5 ml H₂SO₄ in Milli-Q™ H₂O. All carbon and nitrogen sources were added from 0.22 μM filtered stock solutions to 1 g/L. Solid media consisted of the same components with the addition of 20 g/L Difco Bacto Noble Agar.

Cultures for use in enzymatic activity screening were grown in MSB medium (Stanier et al., 1957) with DL-β-phenylalanine added at 2 g/L. Isolates unable to grow in MSB were grown in Nutrient Broth with DL-β-Phenylalanine at 2 g/L. MSB medium comprised 40 ml/L Solution A, 20 ml/L Solution B, 5 ml/L Solution C in Milli-Q™ H₂O. The pH of the medium was 7.2. Carbon sources were added from sterile solutions. Solution A comprised 141.2 g/L Na₂HPO₄, and 136.0 g/L KH₂PO₄ in Milli Q H₂O. Solution B comprised 10 g/L nitrilotriacetic acid, 29.3 g/L MgSO₄·7H₂O, 3.33 g/L CaCl₂·2H₂O, 0.00925 g/L (NH₄)₆Mo₇O₂₄·4H₂O, 0.099 g/L FeSO₄·7H₂O, 50 ml/L Metals 44 Solution, with a few drops of H₂SO₄, in Milli-Q™ H₂O. Solution C comprised 60 g (NH₄)₂SO₄ in 0.3 L Milli Q H₂O. Metals 44 Solution comprised 0.25 g EDTA, 0.1095 g ZnSO₄·7H₂O, 0.154 g MnSO₄·H₂O, 0.5 g FeSO₄·7H₂O, 0.0392 g CuSO₄·5H₂O, 0.0248 g Co(NO₃)₂·6H₂O, 0.0177 g Na₂B₄O₇·10H₂O, 2 drops of H₂SO₄, in 100 ml Milli Q H₂O.

Nutrient medium was prepared from Difco Bacto Nutrient Broth or Difco Bacto Nutrient Agar as described by the manufacturer. Yeast Malt medium was prepared from Difco Bacto YM Broth for liquid cultures, or with 20 g/L Difco Bacto Agar for solid medium.

Scale up conditions used in preparation of cell mass for protein purification work were determined from shake flask studies in MSB medium with various supplements. Ultimately conditions for 10 liter fermentations were 30° C., pH 7.0, 400 rpm agitation, dissolved oxygen set to 100% and maintained above 30%, 500 mB pressure, glucose control between 1 and 2 g/L, and typically 125 mg (R,S)-β-phenylalanine added every 10 hours elapsed fermentation time. Batch conditions comprised 5.65 g/L K₂HPO₄, 5.44 g/L KH₂PO₄, 2 g/L DL-β-phenylalanine, 5 g/L Yeast Extract, 10 mL UCON LB625 antifoam. Post sterilization at 121° C. and cooling for 30 minutes, 10 ml SR-005 10% Trace Metals Solution, 10 ml SR-001 (4.0 mg/ml) CaCl₂ Solution, 10 ml SR-002 (0.3g/ml) MgSO₄ Solution, and 10 g/L SR-008 (50%) Glucose Solution were added. SR-005 Trace Metals Solution comprised 0.5 g/L MnSO₄·H₂O, 0.2 g/L H₃BO₃, 0.8 g/L CuSO₄·5H₂O, 0.7 g/L Na₂MoO₄·2H₂O, 0.7 g/L CoCl₂·6H₂O, 0.4 g/L ZnSO₄·7H₂O, 37.8 g/L FeCl₃·6H₂O, 3.4 ml/L H₂SO₄, in 1L distilled H₂O. Tanks containing 9.5 liters of this medium were inoculated by sterile transfer of overnight 500 ml volume seed cultures of similar medium grown aerobically in baffled 2.8L Fernbach flasks.

Analytical Methods

Protein determinations were made using Bradford reagent and were estimated based on Bovine Serum Albumin protein standard average absorbance vs. concentration. Absorbance readings were acquired using 10×4×45 mm plastic Sarstedt cuvettes in a Hewlett Packard 8453 Spectrophotometer. Procedures supplied by Sigma were followed exactly.

Reverse phase HPLC analysis was performed using a VYDAC 218TP C18, 4.6×150 mm column (The Separations Group, Hesperia, Calif.) on a Hewlett Packard 1100 HPLC system. Separation of substrates and products was achieved using a 10% isocratic elution of HPLC grade methanol containing 0.1% trifluoroacetic acid,(TFA) and 90% MilliQ H₂O containing 0.1% trifluoroacetic acid for 3 minutes. A linear ramp to 40% methanol/0.1% trifluoroacetic acid and 60% MilliQ H₂O/0.1% trifluoroacetic acid followed to 5 minutes. Isocratic elution at 40% methanol/0.1% TFA and 60% MilliQ H₂O/0.1% trifluoroacetic acid continued to 14 minutes. Retention times of β-phenylalanine and 3-keto-3-phenylpropionic acid were 4 and 7.5 minutes, respectively. UV detection was carried out at 254 nM.

Chiral analysis of amino acid enantiomers was performed using an (S,S) Whelk-O1, 4.6×250 mm column (Regis, Morton Grove, Ill.) on a Hewlett Packard 1100 HPLC system. Separation of enantiomers was achieved using an isocratic 60% isopropyl alcohol elution. Amino acids were first esterified by reaction to completeness with absolute ethanol containing 2.52 M HCl. Ester solutions were then evaporated to dryness, and derivatized with 1-naphthoyl chloride. Retention times of the naphthoylated D- and L-β-phenylalanine ethyl esters were 14.7 and 16.7 minutes respectively. UV detection was carried out at 290 nM.

Chiral analysis was also performed using an Eclipse XDB-C8 4.6×150 mm column (Agilent Technologies) on a Hewlett Packard 1100 HPLC system. Samples were derivatized using Marfey's reagent (N-α(2,4-dinitro-5-fluroophenyl) alaninaminde). Diastereomers were separated using a 50% to 100% methanol gradient, with the solvents acidified with 0.1% trifluoroacetic acid. Detection was carried out at 330 nm.

Confirmations of mass and structure were achieved by LC/MS and GC/MS, using standard techniques. The analyses were performed by the Analytical Services Center, Pfizer Corporation.

β-Transaminase Aminating Activity Assays

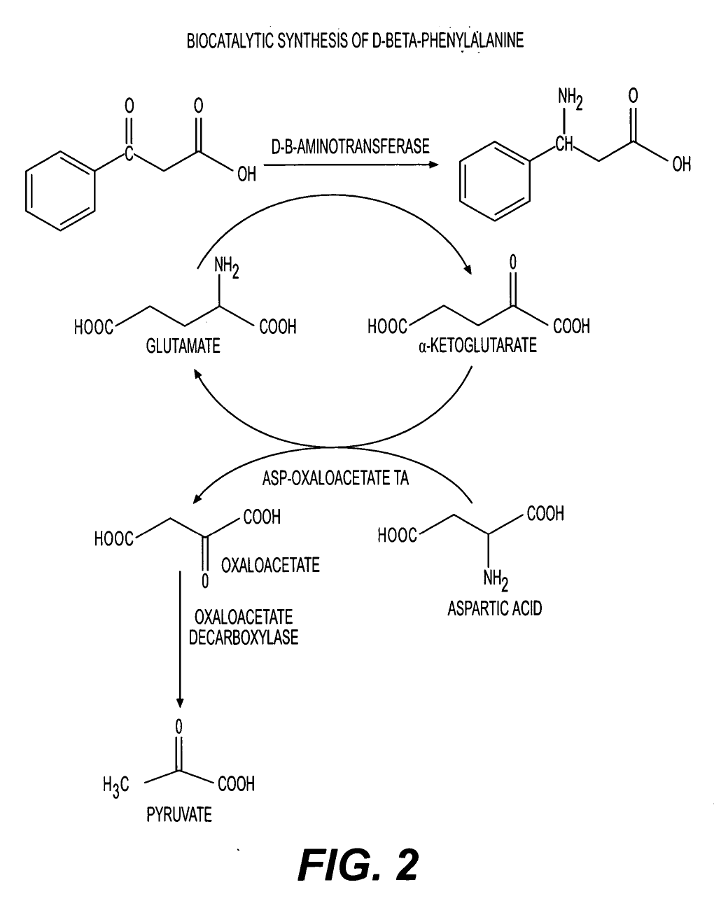

Aminating activity resulting in D-β-Phenylalanine production was assayed by adding 0.1-0.3 mg/ml protein into the following assay system: 100 mM potassium phosphate, 3-keto-3-phenylpropionate (varying concentrations), 20 mM L-glutamic acid, 0.2 mM pyridoxal phosphate, 100 mM L-aspartic acid, 50 U oxaloacetate decarboxylase, 40-50 U glutamic-oxaloacetic transaminase, 10 mM MgCl₂ at pH 8.0. The assays were run at 37° C. with stirring. At predetermined time points, 0.1 ml samples were withdrawn, 0.1 ml 0.1N HCl added, and acidified samples spun in a microcentrifuge at the highest setting for 3 minutes. The supernatant solution was transferred into HPLC sample vials. Conversion of 3-keto-3-phenylpropionate to β-Phenylalanine was quantified using reverse phase HPLC. 3-keto-3-phenylpropionate was prepared from ethyl benzoylacetate using hog liver esterase (Chirazyme E-2) from Roche. β-Phenylalanine resulting from enzymatic transamination of 3-keto-3-phenylpropionic acid was collected from the HPLC, post separation and post detection. Collected volumes were evaporated to dryness. These collected fractions were submitted for mass spectral analysis along with authentic samples, as well as being derivatized for chiral analysis using chiral HPLC. FIG. 2 shows a reaction scheme for the biocatalytic synthesis of D-β-phenylalanine from 3-keto-3-phenylpropionic acid and glutamate in the presence of a stereospecific D-β-aminotransferase.

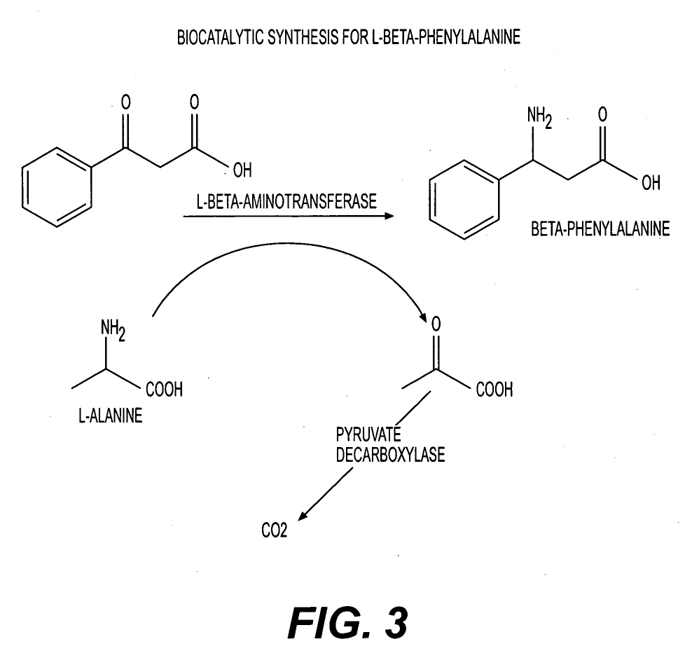

Aminating activity toward L-β-phenylalanine production was assayed by adding 0.1-0.3 mg/ml protein into the following assay system: 100 mM potassium phosphate, 3-keto-3-phenylpropionate (varying concentrations), 20 mM L-alanine, 0.2 mM pyridoxal phosphate, 50 U pyruvate decarboxylase, at pH 8.0. The assays were run at 37° C. with stirring. At predetermined time points, 0.1 ml samples were withdrawn, 0.1 ml 0.1N HCl added, and acidified samples spun in a microcentrifuge at the highest setting for 3 minutes. The supernatant solution was collected into HPLC sample vials. Conversion of 3-keto-3-phenylpropionate to β-phenylalanine was quantified using reverse phase HPLC. 3-keto-3-phenylpropionate was prepared from ethyl benzoylacetate using hog liver esterase (Chirazyme E-2) from Roche. FIG. 3 shows a reaction scheme for the biocatalytic synthesis of L-O-phenylalanine and pyruvate from 3-keto-3-phenylpropionic acid and L-alanine in the presence of a stereospecific L-β-aminotransferase.

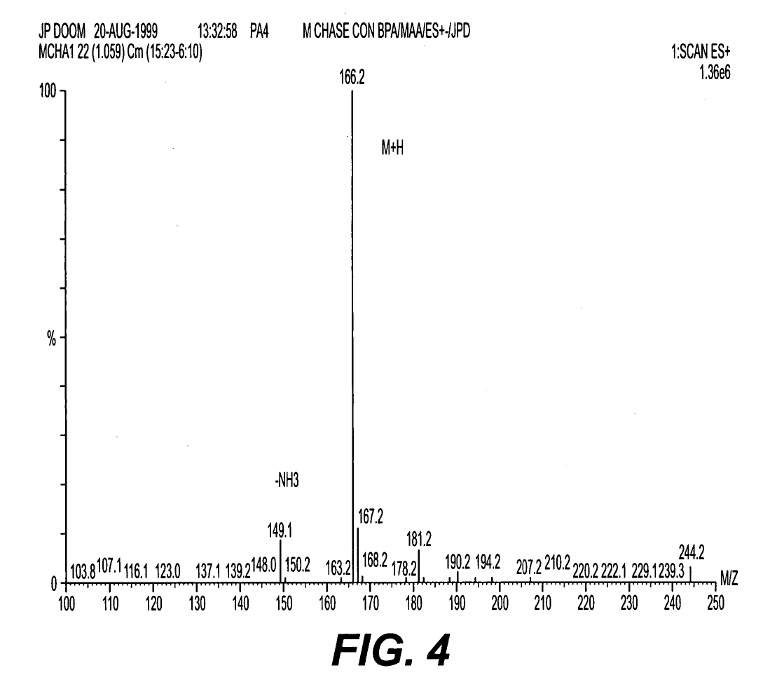

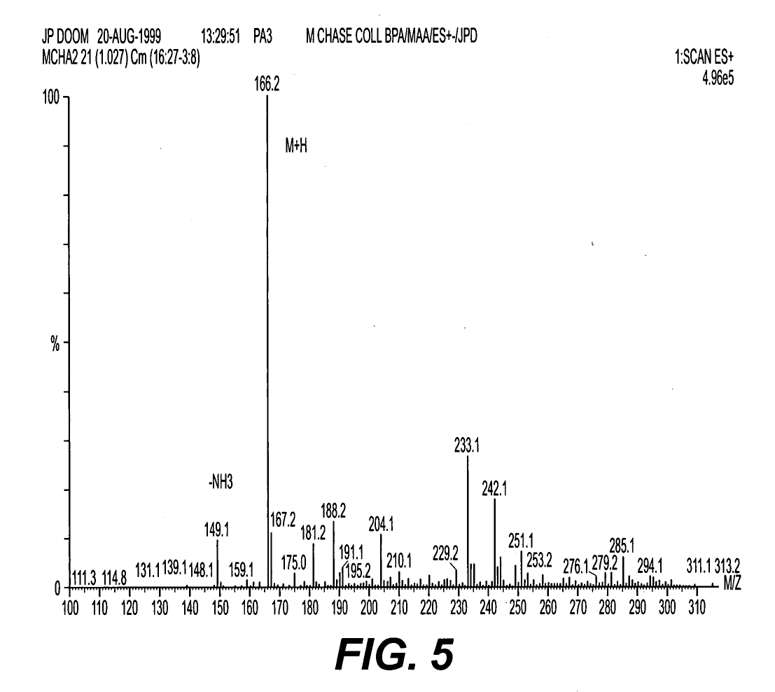

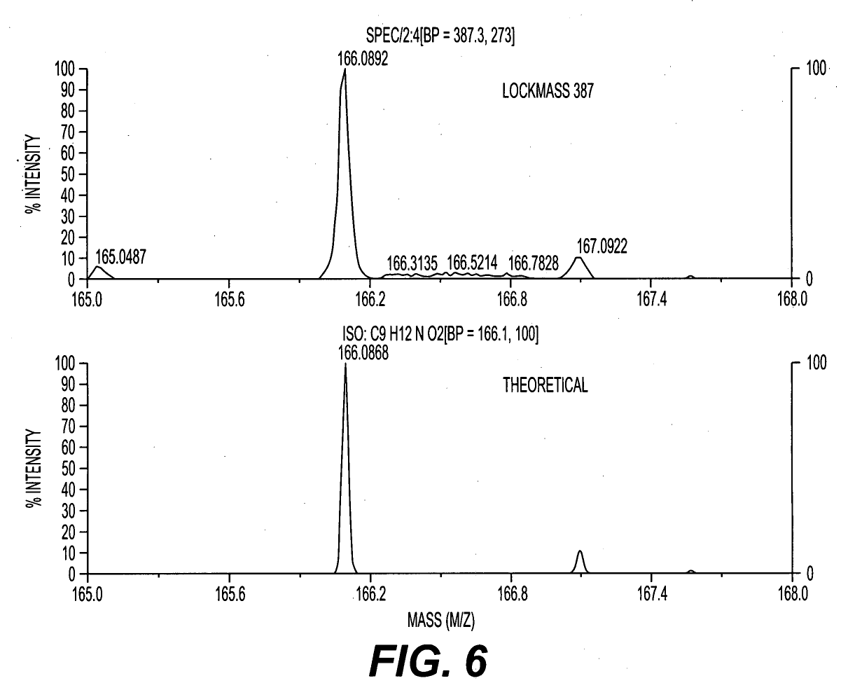

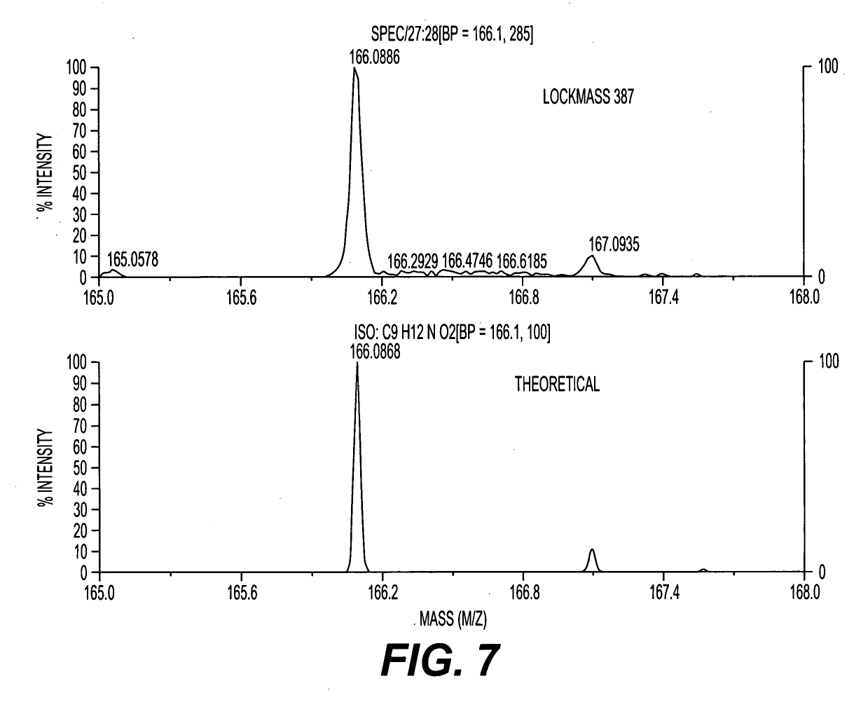

FIGS. 4 and 6 show mass spectral data obtained from authentic DL-β-phenylalanine. HPLC fractions from analyses of enzymatic transamination reactions using the Variovorax paradoxus enzyme were collected and evaporated to dryness. FIGS. 5 and 7 represent mass spectral data obtained from these samples. It is clear from these data that the β-phenylalanine produced biocatalytically using the newly isolated enzyme was structurally identical to authentic β-phenylalanine.

β-Transaminase Deaminating Activity Assay

β-Transaminase deaminating activity was assayed by adding 0.1-0.3 mg/ml protein into the following assay system: 100 mM potassium phosphate, 2 mg/ml DL-β-phenylalanine, 10 mg/ml α-ketoglutarate or pyruvic acid, 0.1 mM pyridoxal phosphate, pH 8.0. The assays were run at 37° C. with stirring. At predetermined time points, 0.1 ml samples were withdrawn, 0.1 ml 0.1N HCl added, and acidified samples spun in a microcentrifuge at the highest setting for 3 minutes. The supernatant solution was transferred into HPLC sample vials. Conversion of DL-β-phenylalanine to 3-keto-3-phenylpropionate was quantified using reverse phase HPLC.

Nucleic Acid Hybridization

As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook, et al., Molecular Cloning, A Laboratory Manual, 2nd Ed., Cold Spring Harbor Press, Cold Spring Harbor, N.Y. (1989), and by Haymes, et al. Nucleic Acid Hybridization, A Practical Approach, IRL Press, Washington, D.C., 1985). Departures from complete complementarity are therefore permissible, as long as such departures do not completely preclude the capacity of the molecules to form a double-stranded structure.

Appropriate stringency conditions which promote DNA hybridization are, for example, 6× sodium saline citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a moderately low stringency of about 2×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Conditions which promote low, medium, and high stringency RNA:DNA or RNA:RNA hybridization reactions, depending on the length of the corresponding nucleic acids, are also well known in the art.

### EXAMPLES

The following examples will illustrate the invention in greater detail, although it will be understood that the invention is not limited to these specific examples. Various other examples will be apparent to the person skilled in the art after reading the present disclosure without departing from the spirit and scope of the invention. It is intended that all such other examples be included within the scope of the appended claims.

### Example 1

### Enrichment Culture and Isolation

Environmental samples from two locations in Foristell, Mo. were collected and stored at 4° C. One sample was collected from an area of close proximity to a chicken pen, or coop, an area likely to have been exposed to high concentrations of nitrogen-rich compounds. Another sample was collected from an area near a compost pile, likely to have been exposed to decaying leaf litter as well as other decaying organic matter. 5 grams of soil sample were inoculated into 50 ml 100 mM KPO₄ buffer, pH 7.0, in 250 ml baffled shake flasks. After being shaken aerobically at 200 rpm and 28° C. for 18 hours, the flask contents were allowed to settle for 2-3 hours.

One ml of each supernatant from the settled soil mixtures was inoculated into 25 mL Slater's enrichment medium in 250-ml baffled shake flasks. Each soil sample was inoculated into six enrichment conditions: 1 mg/ml a-ketoglutarate with 1 mg/ml DL-β-phenylalanine, 1 mg/ml sodium pyruvate with 1 mg/ml DL-β-phenylalanine, 1 mg/ml α-ketoglutarate with no added nitrogen source, 1 mg/ml sodium pyruvate with no added nitrogen source, 1 mg/ml DL-β-phenylalanine, or no additions. All flasks were shaken aerobically at 200 rpm and 28° C. for 6 days.

One ml of each primary flask (“first pass”) was inoculated into 25 ml of the same enrichment medium. These flasks were shaken aerobically at 200 rpm and 28° C. for 48 hours. One ml of each secondary flask (“second pass”) was inoculated into 25 ml of the same enrichment medium. These “third pass” cultures were shaken aerobically at 200 rpm and 28° C. for 48 hours. Each third pass enrichment culture was serially diluted 10-fold to 10⁻⁸ in Slater's enrichment medium minus carbon or nitrogen additions. 100 uL aliquots of each dilution were spread onto solidified Slater's enrichment medium of the same composition as that third pass culture's liquid medium. These agar plates were sealed with Parafilm™ and incubated at 28° C.

Individual representative colonies grown from the third pass enrichments were picked from the enrichment plates using a sterile inoculation loop and transferred onto nutrient agar plates if bacterial or yeast-like in appearance, or onto YM agar plates if fungal in appearance. Each isolate colony picked was given a name corresponding to soil sample origin, carbon and nitrogen source of enrichment, and the number of the order in which the isolate was picked. Plates were sealed with Parafilm™ and incubated at 28° C. overnight. All colonies were streaked for isolation onto nutrient rich agar plates of the same composition. Isolated colonies were harvested and inoculated into 2 ml liquid cultures of the same medium composition and grown aerobically overnight at 28° C. All isolates were then stored as 25% glycerol stocks at 80° C.

### Example 2

### Screening of Microorganisms for Transaminase Activity

Environmental isolates as well as commercially-available microorganisms were screened for transaminase activity on DL-β-phenylalanine. One frozen glycerol vial of each organism was used to inoculate 25 ml MSB liquid medium in 250 ml flasks with 2 g/L DL-β-phenylalanine, or Nutrient Broth with 2 g/L DL-β-phenylalanine in the event that particular strain exhibited poor growth in a minimal medium. Growth of organisms was obtained under aerobic conditions at 28° C. at 1/10 flask volume. Growth of organisms was monitored until the culture was visibly turbid, typically after 24 to 48 hours.

Cultures were divided into two equal volumetric portions. For whole cell bioconversion assays, one half of the culture was pelleted by centrifugation at up to 8000× g for 10 minutes at 4° C. The supernatant was discarded, and the cell mass washed two times with 25 ml of 100 mM potassium phosphate buffer, pH 8.0. The washed cells were then suspended at double the cell concentration of the original culture in transaminase bioconversion assay buffer. Transaminase bioconversion assay buffer consisted of 100 mM potassium phosphate, 10 mg/ml DL-β-phenylalanine, and 10 mg/ml α-ketoglutarate at pH 8.0. The bioconversions were run at 28° C. with 200 rpm shaking for 48 hours. At 48 hours reaction time, 1 ml samples were withdrawn, and cells removed by centrifugation. The supernatant solution was collected into HPLC sample vials. Conversion of DL-β-phenylalanine to 3-keto-3-phenylpropionate was quantified using reverse phase HPLC.

Crude cell homogenates were prepared from the remaining half of the microbial cultures. The cells were pelleted and washed twice in 100 mM potassium phosphate buffer as in the whole cell assay procedure. The washed cell pellets were suspended at three times the pellet volume in French pressure cell breakage buffer. French pressure cell breakage buffer consisted of 100 mM potassium phosphate buffer, pH 8.0, 1 mM dithiothreitol, 1 mM magnesium chloride, 1 mM phenylmethylsulfonyl-flouride (PMSF), and 0.1 mg/ml deoxyribonuclease in Milli-Q H₂O. All manipulations maintained contact with ice. Cells were ruptured by one pass through a French pressure cell at 15,000-20,000 psi. Cell debris was removed by centrifugation in an Eppendorf microcentrifuge for 15 minutes at the highest setting. Supernatant was collected into fresh tubes and placed on ice. Protein concentrations of crude cell homogenates were determined using standard Bradford protein assay conditions. Transaminase activity of the crude cell homogenates was assayed by adding 0.1-0.3 mg/ml protein into the following assay system: 100 mM potassium phosphate, 0.5 mM dithiothreitol, 2 mg/ml DL-β-phenylalanine, 10 mg/ml α-ketoglutarate or sodium pyruvate, 0.1 mM pyridoxal phosphate, pH 8.0. The bioconversions were run at 28° C. with 200 rpm shaking for 48 hours. At 48 hours reaction time, 1 ml samples were withdrawn, and cells removed by centrifugation. The supernatant solution was collected into HPLC sample vials. Conversion of DL-β-phenylalanine to 3-keto-3-phenylpropionate was quantified using reverse phase HPLC.

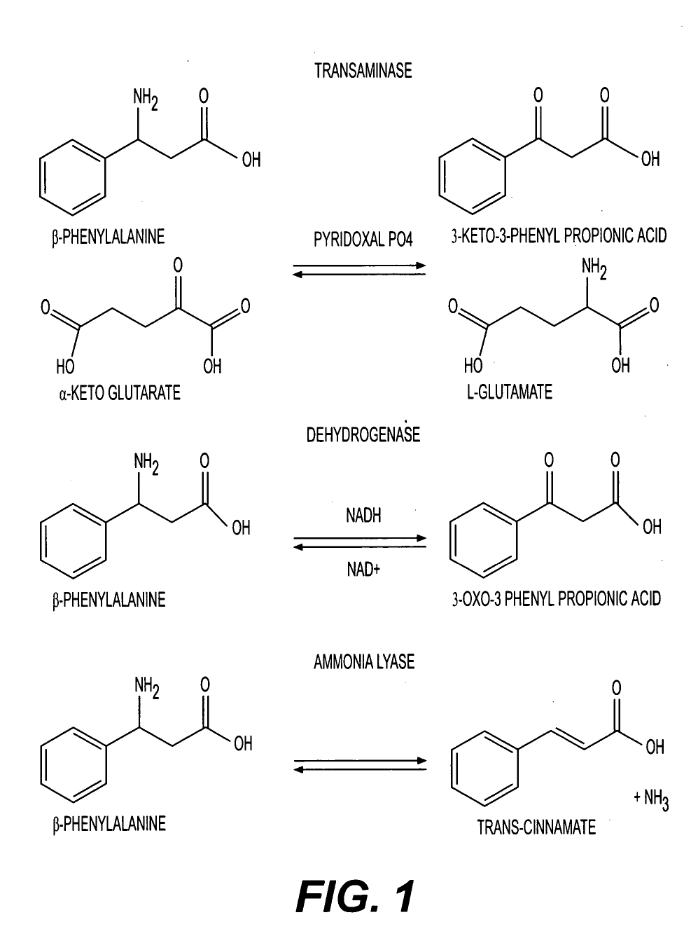

Selective enrichment in media containing DL-β-phenylalanine as a sole source of nitrogen yielded 58 morphologically different soil isolates. Due to the nature of the selective enrichment process employed, it was not possible to determine the enzyme mechanism responsible for growth on β-phenylalanine. Isolates were screened for general deaminase activity on DL-β-phenylalanine using whole cell assays as well as crude cell homogenate enzymatic assays. Possible reaction schemes based on the enrichment mechanism are shown in FIG. 1. The crude cell homogenate assays were targeted toward specifically identifying β-transaminase expressing isolates, while not identifying dehydrogenase and lyase mechanisms. Whole cell assays targeted all three potential mechanisms.

Tables 1 and 2 show data from the crude cell homogenate and the whole cell deaminase assays. The results are reported as 3-keto-3-phenylpropionic acid quantity (peak area at A₂₅₄) per mg protein for crude homogenate assays (Table 1) or per A₆₆₀ for whole cell assays from 48-hour time point reverse phase HPLC analysis (Table 2).

TABLE 1
Deaminase activity in crude cell homogenates

| 3-keto-3-phenylpropionate/ | |
|---|---|
| mg protein | Soil Isolate |
| 70,606.53 | cc-PyrbPhe-I4 |
| 47,110.07 | cc-PyrbPhe-I6 |
| 42,724.91 | cc-bPhe-I1 |
| 40,645.93 | cp-bPhe-I5 |
| 39,722.59 | cp-bPhe-I1 |
| 38,057.08 | cp-KGbPhe-I1 |
| 32,556.72 | cp-bPhe-I7 |
| 31,662.58 | cc-KGbPhe-I14 |
| 29,886.71 | cp-PyrbPhe-I7 |
| 24,478.66 | cc-bPhe-I4 |
| 23,906.75 | cc-PyrbPhe-I3 |
| 21,696.23 | cp-bPhe-I6 |
| 20,588.84 | cp-PyrbPhe-I3 |
| 18,249.54 | cc-bPhe-I5 |
| 15,739.49 | cp-PyrbPhe-I4 |
| 14,873.83 | cp-bPhe-I9 |
| 14,637.93 | cp-bPhe-I3 |
| 14,632.36 | cp-bPhe-I10 |
| 13,143.25 | cp-PyrbPhe-I2 |
| 12,533.87 | cc-bPhe-I6 |
| 526.92 | cp-PyrbPhe-I1 |
| 358.24 | cc-KGbPhe-I12 |
| 311.39 | cc-KGbPhe-I5 |
| 277.11 | cc-KGbPhe-I13 |
| 252.24 | cc-KGbPhe-I1 |
| 183.52 | cc-bPhe-I7 |
| 162.79 | cc-KGbPhe-I2 |
| 155.11 | cc-KGbPhe-I9 |
| 155.08 | cc-KGbPhe-I6 |
| 147.09 | cc-bPhe-I2 |
| 145.79 | cc-PyrbPhe-I8 |
| 138.51 | cc-KGbPhe-I11 |
| 133.39 | cc-KGbPhe-I18 |
| 131.12 | cc-PyrbPhe-I5 |
| 120.05 | cc-KGbPhe-I8 |
| 118.50 | cc-KGbPhe-I7 |
| 116.78 | cp-bPhe-I2 |
| 88.20 | cc-bPhe-I5 |
| 82.47 | cp-bPhe-I4 |
| 71.48 | cp-KGbPhe-I3 |
| 68.25 | cp-PyrbPhe-I6 |
| 66.41 | cc-KGbPhe-I4 |
| 57.73 | cc-KGbPhe-I3 |
| 54.85 | cc-PyrbPhe-I1 |
| 54.05 | cp-KGbPhe-I6 |
| 53.91 | cp-KGbPhe-I4 |
| 41.60 | cc-PyrbPhe-I7 |
| 40.28 | cc-KGbPhe-I10 |
| 33.21 | cc-KGbPhe-I17 |
| 31.42 | cc-KGbPhe-I15 |
| 25.73 | cp-KGbPhe-I2 |
| 20.46 | cc-KGbPhe-I16 |
| 12.07 | cc-PyrbPhe-I5 |
| 8.06 | cp-PyrbPhe-I8 |
| 0.00 | cc-PyrbPhe-I2 |

TABLE 2
Deaminase activity in whole cell preparations

| 3-keto-3-phenylpropionate/OD | Soil Isolate |
|---|---|
| 1,145.96 | cp-PyrbPhe-I7 |
| 191.17 | cc-bPhe-I6 |
| 85.12 | cc-bPhe-I1 |
| 82.82 | cc-KGbPhe-I8 |
| 71.25 | cc-KGbPhe-I6 |
| 55.76 | cc-KGbPhe-I12 |
| 44.09 | cc-KGbPhe-I10 |
| 32.10 | cc-KGbPhe-I5 |
| 25.21 | cp-KGbPhe-I2 |
| 17.68 | cc-KGbPhe-I18 |
| 12.57 | cc-bPhe-I5 |
| 9.42 | cc-PyrbPhe-I7 |
| 7.86 | cp-PyrbPhe-I5 |
| 6.87 | cp-PyrbPhe-I8 |
| 6.46 | cc-PyrbPhe-I5 |
| 6.09 | cc-bPhe-I2 |
| 5.69 | cc-KGbPhe-I3 |
| 5.53 | cc-bPhe-I5 |
| 5.00 | cp-PyrbPhe-I2 |
| 4.71 | cc-KGbPhe-I11 |
| 4.54 | cc-KGbPhe-I7 |
| 2.20 | cc-KGbPhe-I9 |
| 2.01 | cc-KGbPhe-I15 |
| 1.78 | cc-PyrbPhe-I8 |
| 1.65 | cc-KGbPhe-I13 |
| 1.64 | cc-bPhe-I4 |
| 1.14 | cc-PyrbPhe-I2 |
| 1.03 | cc-PyrbPhe-I4 |
| 0.91 | cc-KGbPhe-I17 |
| 0.87 | cp-PyrbPhe-I6 |
| 0.85 | cc-PyrbPhe-I3 |
| 0.84 | cp-KGbPhe-I6 |
| 0.77 | cp-bPhe-I2 |
| 0.65 | cc-PyrbPhe-I1 |
| 0.63 | cc-bPhe-I8 |
| 0.52 | cp-PyrbPhe-I4 |
| 0.48 | cc-KGbPhe-I4 |
| 0.47 | cp-bPhe-I8 |
| 0.04 | cc-PyrbPhe-I6 |
| 0.00 | cc-KGbPhe-I16 |
| 0.00 | cc-KGbPhe-I14 |
| 0.00 | cp-KGbPhe-I3 |
| 0.00 | cc-bPhe-I7 |
| 0.00 | cp-KGbPhe-I1 |
| 0.00 | cp-PyrbPhe-I1 |
| 0.00 | cp-KGbPhe-I4 |
| 0.00 | cp-PyrbPhe-I3 |
| 0.00 | cp-bPhe-I4 |
| 0.00 | cp-bPhe-I1 |
| 0.00 | cp-bPhe-I5 |
| 0.00 | cp-bPhe-I7 |
| 0.00 | cp-bPhe-I6 |
| 0.00 | cp-bPhe-I3 |
| 0.00 | cp-bPhe-I9 |
| 0.00 | cp-bPhe-I10 |

The activities of the different isolates clearly differ when crude cell homogenates were used as a source of enzyme. In many cases, however, differences were noted between results obtained when the enzyme was prepared from whole cells compared to crude cell homogenates. Isolate cp-PyrbPhe-I6, for example, had the highest activity on (R,S)-β-phenylalanine when assayed as a crude cell homogenate compared to all other isolates. This isolate, in a whole cell assay, had one of the lowest deaminase activities on DL-β-phenylalanine, compared to the other isolates.

### Example 3

### Microbial Identification by GC-FAME and BIOLOG Analysis

Taxonomic identification of purified microorganisms was performed by comparing the profiles of fatty acids in each organism with known microorganisms (GC-FAME analysis) and the pattern of growth in a variety of different carbon sources (BIOLOG analysis). Microbial identifications made were based on comparison of GC-FAME and BIOLOG experimental data with state-of-the-art databases, as described below (Microbe Inotech Laboratories, Inc., St. Louis, Mo.). The use of GC-FAME and BIOLOG data to facilitate the taxonomic identification and classification of unknown microorganisms has been compared (Barnett, S. J.; Alami, Y.; Singleton, I.; Ryder, M. H. Diversification of Pseudomonas corrugata 2140 produces new phenotypes altered in GC-FAME, BIOLOG, and in vitro inhibition profiles and taxonomic identification. Can. J. Microbiol. (1999), 45(4), 287-298).

BIOLOG is a commercially-available automated, metabolic characterization system for the identification of microorganisms. The system consists of a 96 well microtiter plate arrayed with a variety of carbon sources and metabolism indicator. Tetrazolium dye in each of the wells turns darker shades of purple as the carbon sources are oxidized by the microorganism. The test yields a characteristic pattern of positive (purple) and negative wells which provide a metabolic signature of each inoculated organism. The pattern of activity toward substrates is compared to that of known microorganisms and, based on matching a known profile, identification is established. Several databases are used to facilitate the comparison, including the metabolic profiles from aerobes, anaerobes, gram positive and negative bacteria, yeast, actinomycetes, and lactic acid bacteria (www.biolog.com/bacteriaid.htm).

Gas-Chromatography of Fatty Acid Methyl Esters (GC-FAME), or cellular fatty acid analysis, is an effective tool for the identification of microbes important in industrial and clinical applications. GC-FAME is a method for identification of yeasts, fungi, anaerobic and aerobic bacteria, mycobacteria, and actinomycetes based on the unique composition of fatty acids of the cell wall (www.microbeinotech.com). Fatty acids (9 to 20 carbon chains long) are extracted from cultured samples and converted to the corresponding methyl esters. The methyl esters are subjected to separation by gas chromatography and the types and concentration of each fatty acid present are recorded. The chromatographic profile is compared through pattern recognition programs to microbial databases containing data collected from over 2600 species and subspecies. The computer-generated reports include the fatty acid profiles, a listing of the best database matches, along with an assigned statistical probability value indicating the confidence level of the match.

The GC-FAME and BIOLOG databases used to identify unknown organisms, represent data collected from thousands of species of bacteria. Each species within a database represents data collected hundreds of samples which are averaged together to determine a set of characteristics unique to each species.

The Similarity and Distance Coefficients refers to the similarity and distance to the hypothetical “mean” organism in the database. A mean database organism has a similarity coefficient of one and a distance of zero. The closer a strain is to one and zero, respectively, the more closely it matches the mean organism in the database. A good match is one with a similarity coefficient greater than 0.5 and a distance coefficient of less than 7.

Eleven soil isolates exhibiting the highest β-transaminase activity on β-phenylalanine relative to the other isolates were selected for identification. Freshly streaked nutrient agar plates were submitted to Microbe Inotech Laboratories, Inc, St. Louis, Mo. Microbial identifications made were based on comparison of GC-FAME and BIOLOG experimental data with state-of-the-art databases. Table 3 shows the results received from Microbe Inotech Laboratories, Inc. Of the eleven strains submitted for typing, there were five clearly distinct genera, and six different genus and species designations.

TABLE 3
Designated Genus Species Names for 11 Soil Isolates

| | Genus Species |
|---|---|
| Isolate Designation | Name Assigned |
| CC-Pyrbphe-I6 | Nocardia asteroides |
| CC-Pyrbphe-I4 | Comamonas terrigena |
| CC-bphe-I1 | Variovorax paradoxus |
| CP-Kgbphe-I1 | Variovorax paradoxus |
| CC-Kgbphe-I14* | Variovorax paradoxus |
| | GC subgroup A |
| CC-bphe-I4 | Variovorax paradoxus |
| CC-Pyrbphe-I3 | Pseudomonas mendocina |
| CP-Pyrbphe-I3 | Variovorax paradoxus |
| CC-bphe-I5 | Variovorax paradoxus |
| CP-Pyrbphe-I4 | Comamonas acidovorans |
| CP-Pyrbphe-I2* | Alcaligenes eutrophus |

*Selected for detailed characterization.

Table 4 summarizes the results obtained by GC-FAME and BIOLOG analysis of the 11 soil isolates. The identity of several isolates could not be established by BIOLOG analysis.

TABLE 4
Summary of Taxonomic Identification by GC-FAME and BIOLOG
analysis

| | Primary ID | | | | | | |
|---|---|---|---|---|---|---|---|
| Strain | by GC- | Sim. | Dif. | Primary ID | Plate | Sim. | Dist |
| No. | FAME | Coef. | Coef. | by BIOLOG | Type | Coef | Coef. |
| 5897-1 cc- | Nocardia | 0.032 | 9.316 | No ID Made | — | — | — |
| PyrbPhe-i6 | asteroids GC | | | | | | |
| | subgroup A | | | | | | |
| 5897-2 cc- | No Match | — | 13.828 | Comamonas | GN | 0.542 | 7.230 |
| PyrbPhe-i4 | Closest: | | | terrigena | | | |
| | Pseudomonas | | | | | | |
| | putida | | | | | | |
| | biotype B | | | | | | |
| 5897-3 cc- | No Match | — | 15.036 | Variovorax | GN2 | 0.600 | 6.220 |
| bPhe-i1 | Closest: | | | paradoxus | | | |
| | Xenorhabdus | | | [Vers.4.0 | | | |
| | nematophilus | | | database] | | | |
| 5897-4 cp- | No Match | — | 13.242 | Variovorax | GN | 0.645 | 5.457 |
| kgbPhe-i1 | Closest: | | | paradoxus | | | |
| | Pseudomonas | | | | | | |
| | putida | | | | | | |
| | biotype B | | | | | | |
| 5897-5 cc- | Variovorax | 0.436 | 4.575 | Variovorax | GN | 0.719 | 4.260 |
| KgPhe-i4 | paradoxus | | | paradoxus | | | |
| | GC subgroup A | | | | | | |
| 5897-6 cc- | No Match | — | 23.591 | Variovorax | GN | 0.601 | 5.462 |
| bPhe-i4 | Closest: | | | paradoxus | | | |
| | Streptococcus | | | | | | |
| | sanguis | | | | | | |
| 5897-7 cc- | Pseudomonas | 0.856 | 19.82 | Genus ID | GN | 0.481 | 7.352 |
| PyrbPhe-i3 | mendocina | | | only: | | | |
| | | | | Acinetobacter | | | |
| | | | | genospecies | | | |
| | | | | 15 | | | |
| 5897-8 cp- | Pseudomonas | 0.297 | 5.877 | Variovorax | GN | 0.724 | 4.187 |
| PyrbPhe-i3 | putida | | | aradoxus | | | |
| | biotype A | | | | | | |
| | [Clin] | | | | | | |
| 5897-9 cc- | Pseudomonas | 0.223 | 6.537 | Variovorax | GN | 0.642 | 5.462 |
| bPhe-i5 | putida | | | paradoxus | | | |
| | biotype A | | | | | | |
| | [Clin] | | | | | | |
| 5897-10 | Comamonas | 0.358 | 5.405 | No ID | — | — | — |
| cp- | acidovorans | | | | | | |
| PyrbPhe-i4 | [Clin] | | | | | | |
| 5897-11 | Alcaligenes | 0.759 | 2.635 | No ID | — | — | — |
| cp- | eutrophus | | | | | | |
| PyrbPhe-i2 | | | | | | | |

### Example 4

### Identification of Microbes by 16S rRNA Sequencing

Ribosomal DNA sequencing was also used to facilitate the taxonomic identification of two purified microorganisms (Kolbert, C. P. and D. H. Persing. 1999. Ribosomal DNA sequencing as a tool for identification of bacterial pathogens. C. Opinions in Microbiol. 2:299-305); Patel, J. B., D. G. B. Leonard, X. Pan, J. M. Musser, R. E. Bergman and I. Nachamkin. 2000. Sequence-Based Identification of Mycobacterium Species Using the MicroSeq 500 16S rDNA Bacterial Identification System. J. Clin. Micro. 38 :246-251). The full-length DNA sequence the 16S rRNA genes (rDNA) of two microorganisms were also determined by MIDI Labs (Newark, Del.) and compared to a database of all known 16S rDNA sequences.

Two microorganisms, designated CC-KGbPhe-I14 and CP-PyrbPhe-I4, were submitted to MIDI Labs, Newark, Del. and the full length DNA sequence of the 16S rRNA genes (rDNA) were determined. The sequence of the 16S RNA genes are shown below (Tables 5, 6, and 7).

TABLE 5
Table of Sequences

| SEQ ID NO | Clone Name | Length | Type |
|---|---|---|---|
| 1 | C3537 (I14 con) | 1530 | DNA |
| 2 | C3538 (I4 con) | 1514 | DNA |

TABLE 6
16S rDNA sequence of isolate CC-KGbPhe-I14 (I14.txt)

| | |
|---|---|
| TGGAGAGTTTGATCCTGGCTCAGATTGAACGCTGGCGGCATGCCTTACACATGCAAGTCGAACGGCAGCGC | (SEQ ID NO: 1) |
| GGGAGCAATCCTGGCGGCGAGTGGCGAACGGGTGAGTAATACATCGGAACGTGCCCAATCGTGGGGGATAA | |
| CGCAGCGAAAGCTGTGCTAATACCGCATACGATCTACGGATGAAAGCAGGGGATCGCAAGACCTTGCGCGA | |
| ATGGAGCGGCCGATGGCAGATTAGGTAGTTGGTGrGGTAAAGGCTCACCAAGCCTTCGATCTGTAGCTGGT | |
| CTGAGAGGACGACCAGCCACACTGGGACTGAGACACGGCCCAGACTCCTACGGGAGGCAGCAGTGGGGAAT | |
| TTTGGACAATGGGCGCAAGCCTGATCCAGCCATGCCGCGTGCAGGATGAAGGCCTTCGGGTTGTAAACTGC | |
| TTTTGTACGGAACGAAACGGCCTTTTCTAATAAAGAGGGCTAATGACGGTACCGTAAGAATAAGCACCGGC | |
| TAACTACGTGCCAGCAGCCGCGGTAATACGTAGGGTGCAAGCGTTAATCGGAATTACTGGGCGTAAAGCGT | |
| GCGCAGGCGGTTATGTAAGACAGTTGTGAAATCCCCGGGCTCAACCTGGGAACTGCATCTGTGACTGCATA | |
| GCTAGAGTACGGTAGAGGGGGATGGAATTCCGCGTGTAGCAGTGAAATGCGTAGATATGCGGAGGAACACC | |
| GATGGCGAAGGCAATCCCCTGGACCTGTACTGACGCTCATGCACGAAAGCGTGGGGAGCAAACAGGATTAG | |
| ATACCCTGGTAGTCCACGCCCTAAACGATGTCAACTGGTTGTTGGCAATTCACTTTCTCAGTAACGAAGCT | |
| AACGCGTGAAGTTGACCGCCTGGGGAGTACGGCCGCAAGGTTGAAACTCAAAGGAATTGACGGGGACCCGC | |
| ACAAGCGGTGGATGATGTGGTTTAATTCGATGCAACGCGAAAAACCTTACCCACCTTTGACATGTACGGAA | |
| TTCGCCAGAGATGGCTTAGTGCTCGAAAGAGAACCGTAACACAGGTGCTGCATGGCTGTCGTCAGCTCGTG | |
| TCGTGAGATGTTGGGTTAAGTCCCGCAACGAGCGCAACCCTTGTCATTAGTTGCTACATTCAGTTGGGCAC | |
| TCTAATGAGACTGCCGGTGACAAACCGGAGCAAGGTGGGGATGACGTCAAGTCCTCATGGCCCTTATAGGT | |
| GGGGCTACACACGTCATACAATGGCTGGTACAAAGGGTTGCCAACCCGCGAGGGGGAGCTAATCCCATAAA | |
| ACCAGTCGTAGTCCGGATCGCAGTCTGCAACTCGACTGCGTGAAGTCGGAATCGCTAGTAATCGTGGATCA | |
| GAATGTCACOGTGAATACGTTCCCGGGTCTTGTACACACCGCCCGTCACACCATGGGAGCGGGTTCTGCCA | |
| GAAGTAGTTAGCTTAACCGCAAGGAGGGCGATTACCACGGCAGGGTTCGTGACTGGGGTGAAGTCGTAACA | |
| AGGTAGCCGTATCGGAAGGTGCGGyTGGATCACCTCCTT | |

TABLE 7
16S rDNA sequence of isolate CP-PyrbPhe-I4 (I4.txt)

| | |
|---|---|
| TGGAGAGTTTGATCCTGGCTCAGGACGAACGCTGGCGGCGTGCTTAACACATGCAAGTCGAGCGGTAAGGC | (SEQ ID NO: 2) |
| CCTTCGGGGTACACGAGCGGCGAACGGGTGAGTAACACGTGGGTGATCTGCCCTGCACTTCGGGATAAGCC | |
| TGGGAAACTGGGTCTAATACCGGATATGACCTTCGGCTGCATGGCTGAGGGTGGAAAGGTTTACTGGTGCA | |
| GGATGGGCCCGCGGCCTATCAGCTTGTTGGTGGGGTAATGGCCTACCAAGGCGACGACGGGTAGCCGACCT | |
| GAGAGGGTGACCGGCCACACTGGGACTGAGACACGGCCCAGACTCCTACGGGAGGCAGCAGTGGGGAATAT | |
| TGCACAATGGGCGAAAGCCTGATGCAGCGACGCCGCGTGAGGGATGACGGCCTTCGGGTTGTAAACCTCTT | |
| TCAGCAGGGACGAAGCGAAAGTGACGGTACCTGCAGAAGAAGCACCGGCCAACTACGTGCCAGCAGCCGCG | |
| GTAATACGTAGGGTGCAAGCGTTGTCCGGAATTACTGGGCGTAAAGAGyTCGTAGGCGGTTTGTCGCGTCG | |
| TCTGTGAAAACTCrmrGCTCAACCkyGAGCyTGCAGGCGATACGGGCAGACTTGAGTACTGCAGGGGAGAC | |
| TGGAATTCCTGGTGTAGCGGTGAAATGCGCAGATATCAGGAGGAACACCGGTGGCGAAGGCGGGTCTCTGG | |
| GCAGTAACTGACGCTGAGGArCGAAAGCGTGGGTAGCGAACAGGATTAGATACCCTGGTAGTCCACGCCGT | |
| AAACGGTGGGCGCTAGGTGTGGGTTTCCTTCCACGGGATCCGTGCCGTAGCTAACGCATTAAGCGCCCCGC | |
| CTGGGGAGTACGGCCGCAAGGCTAAAACTCAAAGGAATTGACGGGGGCCCGCACAAGCGGCGGAGCATGTG | |
| GATTAATTCGATGCAACGCGAAGAACCTTACCTGGGTTTGACATATACCGGAAAGCCGTAGAGATACGGCC | |
| CCCCTTGTGGTCGGTATACAGGTGGTGCATGGCTGTCGTCAGCTCGTGTCGTGAGATGTTGGGTTAAGTCC | |
| CGCAACGAGCGCAACCCTTGTCTTATGTTGCCAGCACGTAATGGTGGGGACTCGTAAGAGACTGCCGGGGT | |
| CAACTCGGAGGAAGGTGGGGACGACGTCAAGTCATCATGCCCCTTATGTCCAGGGCTTCACACATGCTACA | |
| ATGGCCrGTACAGAGGGCTGCGAkACCGTGAGGTGGAGCGAATCCCTTAAAGCyGGTCTCAGTTCGGATCG | |
| GGGTCTGCAACTCGACCCCGTGAAGTCGGAGTCGCTAGTAATCGCAGATCAGCAACGCTGCGGTGAATACG | |
| TTCCCGGGCCTTGTACACACCGCCCGTCACGTCATGAAAGTCGGTAACACCCGAAGCCGGTGGCCTAACCC | |
| CTTGTGGGAGGGAGCCGTCGAAGGTGGGATCGGCGATTGGGACGAAGTCGTAACAAGGTAGCCGTACCGCA | |
| AGGTGCGGyTGGATCACCTCCTT | |

These two microorganisms were identified by database matching to known 16S rDNA sequences as Variovorax paradoxus [strain CC-KGbPhe-I14] (SEQ ID NO: 1) and Rhodococcus opacus [strain CP-PyrbPhe-I4] (SEQ ID NO: 2), respectively. Note that GC-FAME analysis had identified the latter strain [CP-PyrbPhe-I4] as Comamonas acidovorans (also known as Delftia acidovorans, Bacterial Nomenclature Up-To-Date, [Approved Lists, Validation Lists], March 2003 DSMZ (Deutsche Sammlung von Microorganismen und Zellculturen GmbH)), and that BIOLOG analysis on the same strain was inconclusive.

### Example 5

### Enzyme Purification

Large amounts of D-β-aminotransferase were prepared from a microorganism identified as Variovorax paradoxus (Table 8), using the procedure outlined below. Identical procedures are used to purify large amounts of L-α-aminotransferase from Alcaligenes eutrophus. The stereospecificity (as a D- or L-β-aminotransferase), substrate specificity (preferred amino donors and amino acceptors), and cofactor requirements are determined for each enzyme preparation using the procedures outlined below.

TABLE 8
Strains with Described β-Aminotransferase Activity

| | | Reference/ |
|---|---|---|
| Designation | Description or Genotype | Source |
| Variovorax | Gram negative bacterium isolated from an | This work |
| paradoxus | environmental soil sample. Identity | |
| | determined by GC-FAME, BIOLOG, and 16S | |
| | rRNA sequencing (SEQ ID NO: 1). | |
| Alcaligenes | Gram negative bacterium isolated from an | This work |
| eutrophus | environmental soil sample. Identity | |
| | determined by GC-FAME, BIOLOG. | |

One 10 L fermentation was used to produce cell mass for protein purification. Cell paste resulting from cell separation post fermentation was suspended in 3 times the cell paste volume of French pressure cell breakage buffer. Cells were resuspended using a tissue homogenizer. Cells were then disrupted using two passes through a microfluidizer at 16,000 psi. Cell debris and unbroken cells in the resulting homogenate were removed by centrifugation for 20 minutes at up to 8000× g. Supernatant from cell homogenization was then stored at 4° C.

Ammonium sulfate precipitation of crude cell homogenate was used as a first step in the enzyme purification. At 4° C., ammonium sulfate was slowly added with stirring to 30% saturation. The 30% ammonium sulfate solution was allowed to stir for approximately 16 hours. Precipitate was removed by centrifugation at 10,000×g for 3 hours at 4° C. Supernatant solution from this 30% ammonium sulfate precipitation was recovered and stored at 4° C.

The transaminase activity was then partially purified from 1500 ml of a filtered (Sartobran P 0.8/0.2 μm filter unit) fermentation extract containing 30% ammonium sulfate ((NH₄)₂SO₄). Three hundred forty ml of this extract were loaded onto a 170 ml bed volume Butyl Sepharose FF column (3.2×19.8 cm) that had been equilibrated with 1.2 M ammonium sulfate 25 mM Tris, pH 8.1. The flow rate was 5 ml/min and loading was followed by a 4-column volume (CV) wash of equilibration buffer. Gradient elution was performed with 10 CV, from 1.2 M (NH₄)₂SO₄, 25 mM Tris, pH 8.1 to 25 mM Tris, pH 8.1. Column chromatography was repeated a total of 4 times with the last loading volume comprising ˜250 ml. Ninety-five fractions of 13 ml each were collected (˜0.1 CV) and assayed for transaminase activity. Two peaks of activity were detected between fractions 35-55 (peak A) and 70-95 (peak B). All peak A fractions were pooled into a single peak A pool (620 ml @ 0.51 mg protein/ml) as well as for a peak B activity pool (670 ml @ 0.2 mg protein/ml). Each pool was ultrafiltered/diafiltered at 20-25 psi using a 50 cm² 10K MWCO Millipore Pellicon XL cellulose membrane and Millipore Labscale TFF system to 2× starting protein concentration and against 20 mM Tris, pH 9.5.

ach single diafiltered pool of activity from the Butyl Sepharose FF column was loaded directly onto a 80 ml bed volume Q Sepharose HP column (2.6×15 cm). Pool A activity only was diluted 1:1 with equilibration buffer (20 mM Tris, pH 9.5) before loading. The column was eluted with a 10 CV gradient from 20 mM Tris, pH 9.5 to 20 mM Tris, 200 mM NaCl, pH 9.5. Ninety 8 ml fractions (0.1 CV) were collected and assayed for transaminase activity. Pool A activity eluted within fractions 55-85 (120 ml). Pool B activity within fractions 65-75 (68 ml). Each Q column activity pool was concentrated to ¼ pool volume and frozen.

Each pool of activity from the Q Sepharose HP column was further purified by size exclusion HPLC using a TSK G3000 SW×1 column (7.8 mm ×30 cm) at 1 m/min using 50 mM Tris, 100 mM NaCl, pH 7.0 mobile phase. Multiple injections of 100 μL each were chromatographed and 0.5 ml fractions collected from 5 or 10 runs total. Total peak collection was performed by collecting HPLC eluant into the same fractions for each run and assayed for transaminase activity. Both A and B activities eluted within fractions 19-21 at approximately 50 K MW.

### Example 6

### Determination of D-β-Aminotransferase Substrate Specificity

Semi-purified β-aminotransferase enzyme from Variovorax paradoxus (CC-KGbPHe-I14) was assayed using D-β-phenylalanine as the amino donor, in an assay consisting of 100 mM potassium phosphate, 2 mg/ml DL-β-phenylalanine, 10 mg/ml α-keto acceptor and 0.1 mM pyridoxal phosphate, at pH 8.0. The reactions were run at 37° C. with mild stirring for 1 hour. At 1 hour reaction time, 0.1 ml samples were withdrawn, acidified with addition of 0.1 ml 0.1N HCl, and centrifuged. The supernatant solution was collected into HPLC sample vials. Conversion of DL-β-phenylalanine to 3-keto-3-phenylpropionate was quantified using reverse phase HPLC. The following keto acids were screened for activity: α-ketoglutaric acid, oxaloacetic acid, pyruvic acid, 3-oxoadipic acid, 2-oxoadipic acid, 1,3-acetonedicarboxylic acid, and 4-ketopimelic acid. The relative rates of conversion of D-β-phenylalanine to 3-keto-3-phenylpropionic acid using the various keto acceptors is reported as percent of the rate using α-ketoglutarate, the experimentally determined preferred acceptor. The relative rates are shown in Table 9.

TABLE 9
D-β-aminotransferase Keto Acid Substrate Specificity

| Keto Acceptor | Relative Activity |
|---|---|
| α-ketoglutarate | 100% |
| Oxaloacetic Acid | 54% |
| Pyruvic Acid | 14% |
| 3-Oxoadipic Acid | 6% |
| 2-Oxoadipic Acid | 0.6% |
| 1,3-Acetonedicarboxylic Acid | 0% |
| 4-Ketopimelic Acid | 0% |

The same preparation of semi-purified β-aminotransferase enzyme from Variovorax paradoxus was then assayed using a-ketoglutarate as the keto acceptor, in an assay consisting of 100 mM potassium phosphate, 10 mg/ml α-ketoglutarate, 2 mg/ml amino donor and 0.1 mM pyridoxal phosphate, at pH 8.0. The reactions were run at 37° C. with mild stirring for 1 hour. At 1 hour reaction time, 0.1 ml samples were withdrawn, acidified with addition of 0.1 ml 0.1N HCl, and centrifuged. The supernatant solution was collected into HPLC sample vials. Conversion of an amino acid to the corresponding ketone was quantified using reverse phase HPLC. The following amino acids (as an amino donor) were screened for activity: α-(D)-phenylalanine, α-(L)-phenylalanine, β-(D)-phenylalanine, β-(L)-phenylalanine, (D)-3-amino-5-phenylpentanoic acid, and (L)-3-amino-5-phenylpentantoic acid. Only β-(D)-phenylalanine and (D)-3-amino-5-phenylpenantoic acid were substrates for the enzyme under these conditions.

### Example 7

### Determination of L-β-Aminotransferase Substrate Specificity

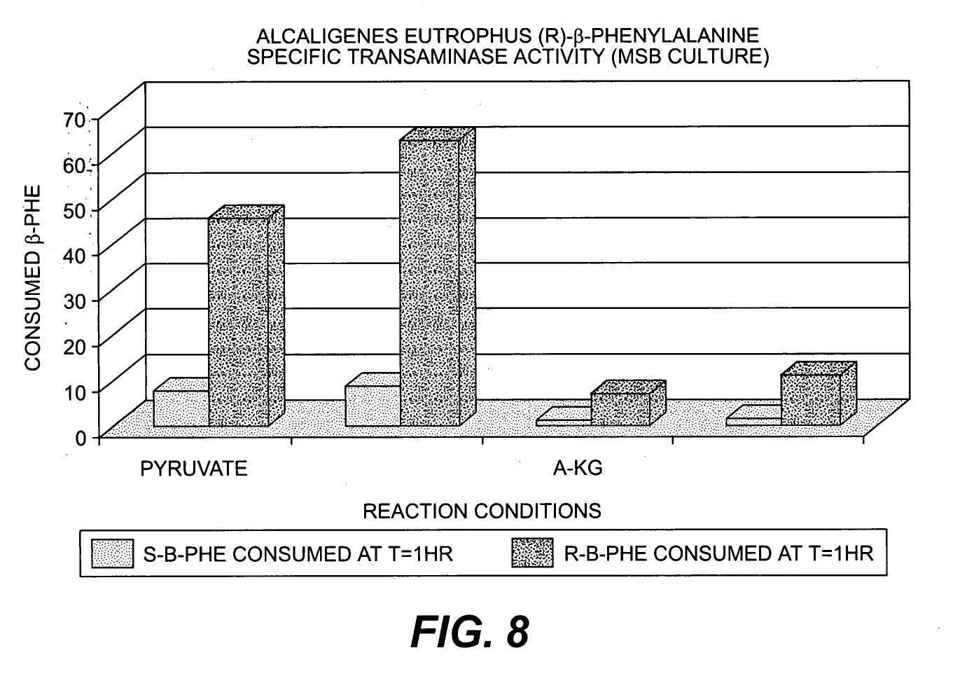

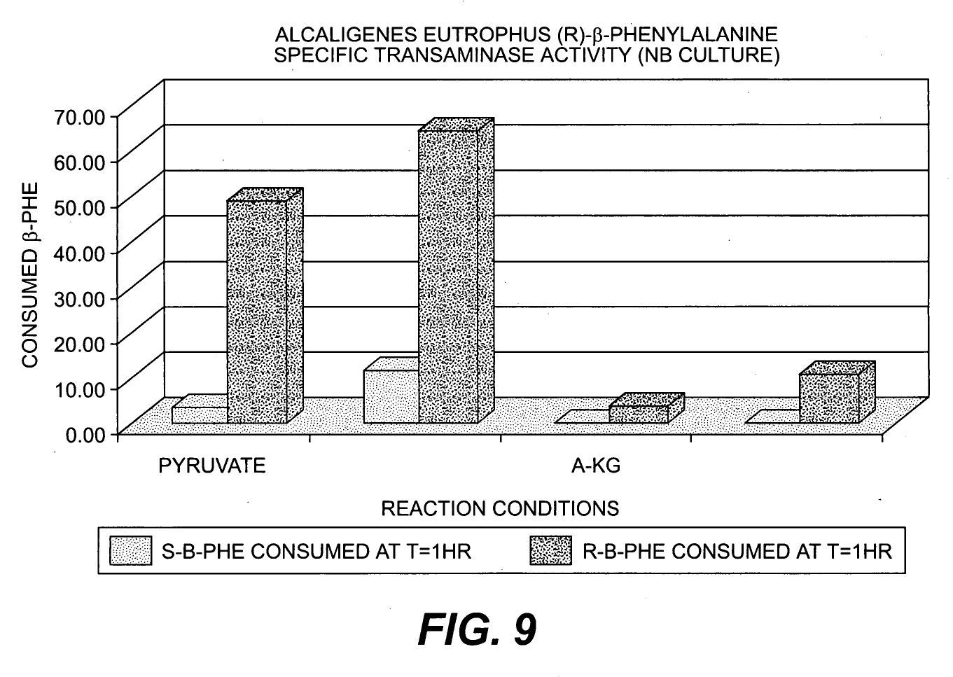

Crude cell homogenate preparations of Alcaligenes eutrophus (CP-PyrbPhe-I2) were made from cells grown in Nutrient Broth supplemented with 1 g/L D,L-β-phenylalanine. Cell-free homogenates were used in assays designed to determine substrate specificity. The assays contained: 0.1-0.3 mg/ml protein, 10 mg/ml keto acceptor, 1 mg/ml amino donor, 0.1 mM pyridoxal phosphate, and 100 mM potassium phosphate, at pH 8.0. The reactions were run at 37° C. with gentle stirring for 1 hour. At 1 hour reaction time, 0.1 ml samples were withdrawn, acidified with addition of 0.1 ml 0.1N HCl, and centrifuged. The supernatant solution was collected into HPLC sample vials. Conversion of amino acid to the corresponding ketone was quantified using reverse phase HPLC. Enzymatic activity was measured as the rate of conversion of amino donor in a one hour period. In a series of reactions, the enzymatic activity was determined with the following variables: inclusion or omission of pyridoxal phosphate, using α-ketoglutarate or pyruvate as a keto acceptor, and (D)- or (L)-β-phenylalanine as amino donor. The L-β-aminotransferase from Alcaligenes eutrophus exhibits a requirement for pyridoxal phosphate, prefers pyruvate as a α-ketoglutarate as a keto acceptor, and uses (L)-β-phenylalanine over the (D)-enantiomer (The data in Table 10 are illustrated graphically in FIG. 8 and FIG. 9).

TABLE 10
Substrate specificity of Alcaligenes eutrophus L-β-aminotransferase

| | | | NB Culture | NB Culture |
|---|---|---|---|---|
| | MSB | MSB | Extract of | Extract of |
| | Culture | Culture | Alcaligenes | Alcaligenes |
| | Extract | Extract | eutrophus | eutrophus |
| | D(S)-b-Phe | L(R)-b-Phe | D(S)-b-Phe | L(R)-b-Phe |
| | consumed | consumed | consumed at | consumed at |
| Conditions | at t = 1 Hr | at t = 1 Hr | t = 1 Hr | t = 1 Hr |
| Pyruvate | 7.789 | 45.1865 | 3.37 | 48.61 |
| Pyruvate/PLP | 8.866 | 62.8012 | 11.40 | 64.14 |
| α-KG | 0.966 | 6.9281 | 0.00 | 3.41 |
| α-KG/PLP | 1.512 | 10.8967 | 0.00 | 10.46 |

In separate assays with this same crude cell homogenate, it was determined that this particular enzyme is able to catalyze the production of β-phenylalanine from 3-keto-3-phenylpropionic acid using L-alanine as an amino donor. The assays contained: 0.1-0.3 mg/ml protein, 10 mg/ml keto acceptor, 200 mM amino donor, 0.1 mM pyridoxal phosphate, and 100 mM potassium phosphate, at pH 8.0. 0.1 units of pyruvate decarboxylase was included to prevent the reversible reaction from reaching equilibrium. The reactions were run at 37° C. with gentle stirring for 24 hours. At 24 hours reaction time, 0.1 ml samples were withdrawn, acidified with addition of 0.1 ml 0.1N HCl, and centrifuged. The supernatant solution was collected into HPLC sample vials. Conversion of amino acid to the corresponding ketone was quantified using reverse phase HPLC. Amino donors screened were (L)-alanine, (D)-alanine, and β-alanine. Only (L)-alanine was utilized by the enzyme as an amino donor in the production of β-phenylalanine.

### Example 8

### Determination of Substrate Specificity in the Stereospecific Synthesis of β-Amino Acids

The substrate specificity the D- and L-β-aminotransferase enzymes isolated from Variouorax paradoxus and Alcaligenes eutrophus, respectively in the stereospecific synthesis of β-amino acid is studied using a variety of amino donors (glutamic acid, aspartic acid, and/or alanine) in the presence of an amino acceptor (selected from Table 11) to form a corresponding β-amino acid product (using the methods described in Examples 8 and 9, above).

TABLE 11
Testing the Substrate Specificity during the Stereospecific
Synthesis of a β-Amino Acid Using a Variety of Amino Donors and
Acceptors

| Amino donor | Amino acceptor | Amino acid product |
|---|---|---|
| Glutamic acid | 3-(2-fluorophenyl)3- | 2-fluoro-β-phenylalanine |
| | oxopropanoic acid | |
| or | 3-(4-fluorophenyl)3- | 4-fluoro-β-phenylalanine |
| | oxopropanoic acid | |
| Aspartic acid | 3-(3-nitrophenyl)3- | 3-nitro-β-phenylalanine |
| | oxopropanoic acid | |
| or | 3-(4-nitrophenyl)3- | 4-nitro-β-phenylalanine |
| | oxopropanoic acid | |
| Alanine | 3-(4-methoxyphenyl)3- | 4-methoxy-β-phenylalanine |
| | oxopropanoic acid | |
| | 3-(4-phenyl)3-oxopropanoic | 4-phenyl-β-phenylalanine |
| | acid | |
| | 3-(2-naphthyl)3-oxopropanoic | 3-amino-3-(2- |
| | acid | naphthyl)propanoic acid |
| | 3-(3-indolyl)oxopropanoic acid | 3-(3-indolyl)aminopropanoic acid |
| | 3-(2-furyl)oxopropanoic acid | 3-(2-furyl)aminopropanoic |
| | | acid |
| | 3-(3-pyridyl)oxopropanoic | 3-(3-pyridyl)aminopropanoic |
| | acid | acid |
| | β-oxo-2-thiophenepropanoic | β-amino-2- |
| | acid | thiophenepropanoic acid |
| | β-oxo-4-bromo-2- | β-amino-4-bromo-2- |
| | thiophenepropanoic acid | thiophenepropanoic acid |
| | 3-oxo-3-(3- | 3-amino-3-(3- |
| | quinolinyl)propanoic acid | quinolinyl)propanoic acid |
| | 3-cyclohexyl-3-oxopropanoic | 3-cyclohexyl-3- |
| | acid | aminopropanoic acid |
| | 3-cyclopropyl-3-oxopropanoic | 3-cyclopropyl-3- |
| | acid | aminopropanoic acid |
| | cyclohexanone-2-carboxylic | 2-aminocyclohexane |
| | acid | carboxylic acid |
| | Cyclopentanone-2-carboxylic | 2-aminocyclopentane |
| | acid | carboxylic acid |
| | acetoacetic acid | 3-aminobutyric acid |
| | Propionylacetic acid | 3-aminopentanoic acid |
| | Butyrylacetate | 3-aminohexanoic acid |
| | 3-oxo-5-cyclopentylpentanoic | 3-amino-5- |
| | acid | cyclopentylpentanoic acid |
| | 3-oxo-5-methylhexanoic acid | 3-amino-5-methylhexanoic |
| | | acid |
| | 3-oxo-3-(3-tetrahydrofuryl) | 3-amino-3-(3-tetrahydrofuryl) propanoic |
| | propanoic acid | acid |
| | 3-oxo-4,4,5,5,6,6,6- | 3-amino-4,4,5,5,6,6,6- |
| | heptafluorohexanoic acid | heptafluorohexanoic acid |
| | 3-oxo-4,4-dimethyl-5- | 3-amino-4,4-dimethyl-5- |
| | chloropentanoic acid | chloropentanoic acid |
| | 3-oxo-4-thiapentanoic acid | 3-amino-4-thiapentanoic acid |
| | 3-oxo-4-oxa-8-chlorooctanoic | 3-amino-4-oxa-8- |
| | acid | chlorooctanoic acid |
| | 3-oxo-5-oxahexanoic acid | 3-amino-5-oxahexanoic acid |
| | 2-ethylacetoacetic acid | 2-ethyl-3-aminobutyric acid |
| | 2-methyl-benzylacetic acid | 2-methyl-β-phenylalanine |
| | 2,2-dimethyl-benzylacetic | 2,2-dimethyl-β- |
| | acid | phenylalanine |

### Example 9

### Determination of Substrate Specificity in the Enantiomeric Enrichment β-Amino Acids

The utility of D- and L-β-aminotransferase enzymes to facilitate the enantiomeric enrichment of a β-amino acid from a racemic mixture of β-amino acids is studied using a variety of amino acceptors (α-keto glutaric acid, oxaloacetic acid, and/or pyruvic acid) in the presence of an amino acceptor (selected from Table 12) to form a corresponding β-amino acid product (using the methods described in Examples 8 and 9, above).

TABLE 12
Testing the Substrate Specificity During Enantiomeric Enrichment
of a β-Amino Acid from a Racemic Mixture of Corresponding
β-Amino Acids

| Amino Acceptor | Racemic Amino Acid Donors |
|---|---|
| α-ketoglutaric acid | 2-fluoro-β-phenylalanine |
| or | 4-fluoro-β-phenylalanine |
| oxaloacetic acid | 3-nitro-β-phenylalanine |
| or | 4-nitro-β-phenylalanine |
| pyruvic acid | 4-methoxy-β-phenylalanine |
| | 4-phenyl-β-phenylalanine |
| | 3-amino-3-(2-naphthyl)propanoic acid |
| | 3-(3-indolyl)aminopropanoic acid |
| | 3-(2-furyl)aminopropanoic acid |
| | 3-(3-pyridyl)aminopropanoic acid |
| | β-amino-2-thiophenepropanoic acid |
| | β-amino-4-bromo-2-thiophenepropanoic acid |
| | 3-amino-3-(3-quinolinyl)propanoic acid |
| | 3-cyclohexyl-3-aminopropanoic acid |
| | 3-cyclopropyl-3-aminopropanoic acid |
| | 2-aminocyclohexane carboxylic acid |
| | 2-aminocyclopentane carboxylic acid |
| | 3-aminobutyric acid |
| | 3-aminopentanoic acid |
| | 3-aminohexanoic acid |
| | 3-amino-5-cyclopentylpentanoic acid |
| | 3-amino-5-methylhexanoic acid |
| | 3-amino-3-(3-tetrahydrofuryl) propanoic acid |
| | 3-amino-4,4,5,5,6,6,6-heptafluorohexanoic acid |
| | 3-amino-4,4-dimethyl-5-chloropentanoic acid |
| | 3-amino-4-thiapentanoic acid |
| | 3-amino-4-oxa-8-chlorooctanoic acid |
| | 3-amino-5-oxahexanoic acid |
| | 2-ethyl-3-aminobutyric acid |
| | 2-methyl-β-phenylalanine |
| | 2,2-dimethyl-β-phenylalanine |

### Example 10

### Resolution of Substrates Using Variovorax paradoxus β-Aminotransferase

Cell Lysate Preparation

Medium Preparation

Nutrient broth (NB), the base medium, was prepared by dissolving 8 grams per liter of deionized water, adding 100 ul UCON LB625 polyalkylene glycol antifoam/liter, and steam sterilizing. A stock solution was prepared by dissolving 1 gram of racemic β-phenylalanine per 20 ml deionized water, while stirring and adding 2N NaOH, to keep the pH at about 10. After dissolution, the pH of the stock solution was decreased to 8 by the addition of 2N HCl, and filter sterilized by passing through a sterile 0.8 micron over 0.2 micron filter. The sterilized filtrate was then added to the sterilized nutrient broth kept at ambient room temperature to a final concentration of 2-3 grams per liter β-phenylalanine.

Culture Growth

Variovorax paradoxus was grown in nutrient broth supplemented with β-phenylalanine. Typically, 500 ml of β-phenylalanine-supplemented nutrient broth were added to a 2.8L Fernbach flask fitted with a silicone sponge foam closure. The culture was inoculated and shaken at 200 rpm with 2 inch strokes at 26° C. until it was fully grown.

Culture Harvest

Cells were harvested by centrifugation for 10 minutes at about 24,000× g. The supernatant was decanted, and because the cell pellet was not very solid, the remaining clear fluid was withdrawn by pipet. The cells were washed by resuspending them in their approximately original volume with 50 mM pH 7.0 phosphate buffer containing 1% NaCl (w/v). The centrifugation and supernatant removal were repeated. The resulting cell pellet was then resuspended in about 35 ml of the same buffer and the suspension centrifuged for 10 minutes at 43,000× g to produce a firm cell pellet. The supernatant was discarded. About 5 ml of the buffer used for resuspension was added to the cell pellet, and the buffer and cell pellet were agitated to produce a cell slurry.

Cell Breakage

The cell slurry was subjected to lysis by means of an Avestin device operated at 20,000 psi. A small amount of DNAseI was added to the suspension prior to loading the Avestin device. The cell slurry was sent through the device once and, based on a non-quantitative microscopic assessment, good lysis was achieved.

Cell-Free Lysate Preparation

The broken cell slurry was centrifuged for 10 minutes at 43,000×g. The clear supernatant was removed and retained, and the remaining slurry was centrifuged again to produce additional clear supernatant. Both clear supernatants were combined, and the pH was adjusted, while stirring, from 6.4 to 8.0 with 2N NaOH. The pH-adjusted supernatant was used fresh or after freezing and thawing up to two times.

Reaction Buffer Preparation

A buffer consisting of 0.3M Tricine, 15 mM MgSO4, enough pyridoxal phosphate to impart a pale yellow color and 0.3M sodium glutamate (for deamination reactions) or 0.3 M 2-ketoglutarate monopotassium salt (for synthesis reactions) was prepared.

Synthesis or Deamination Reactions with Variovorax paradoxus

One part buffer was combined with two parts lysate and this mixture was then added to pre-weighed substrate to give a concentration of 1 mg/ml substrate. Reactions were typically run at 1 ml scale with gentle shaking at 26° C.

Reaction Sample Processing

10 ul of 2N HCl was added to a plastic screw capped vial and 90ul of sample was then added. The pH of the mixture confirmed to be below 4 and the mixture was centrifuged in a microcentrifuge at maximum speed for about 2 minutes to remove precipitated materials.

Samples were derivatized using Marfey's reagent, N•-(2,4-dinitro-5-fluorophenyl)-L-alaninamide. A stock solution of 10 mg/mL in acetone of this reagent was prepared. Either 20 or 40 ul of bioconversion sample was added to 20 ul 1.3 M KHCO₃. To this was added 80 ul of the stock reagent solution, the vial sealed tightly, and heated at 70° C. for 10 minutes. As needed, after cooling to room temperature, the heated samples were centrifuged to remove solids. The supernatants were analyzed using an Eclipse XDB-C8 column, 4.6×150 mm, eluting with methanol gradient in water, from 50% methanol to 100% methanol. Both solvents were acidified with 0.1% trifluoroacetic acid.

For resolution experiments, the area of the peak consumed (converted to keto acid) was compared to the area remaining of the unconsumed peak, on the assumption that none was converted.

For synthesis experiments, in which β-keto acid was converted to β-amino acid, the area of the peak(s) was compared to a standard curve derived for β-phenylalanine.

Individual experiments measuring resolution or synthesis of beta amino acids are described below. The results for the resolution experiments are summarized in Table 13.

Experiment 1

Using the protocol described above for resolution, β-phenylalanine was incubated with cell lysate from Variovorax paradoxus for 23 hours, after which time 82% of the later eluting peak was consumed.

Experiment 2

Using the protocol described above for resolution, p-methoxy-β-phenylalanine was incubated with cell lysate from Variovorax paradoxus for 23 hours, after which time 80% of the later eluting peak was consumed.

Experiment 3

Using the protocol described above for resolution, m-nitro-β-phenylalanine was incubated with cell lysate from Variovorax paradoxus for 23 hours, after which time 95% of the later eluting peak was consumed.

Experiment 4

Using the protocol described above for resolution, p-fluoro-β-phenylalanine was incubated with cell lysate from Variovorax paradoxus for 23 hours, after which time 82% of the later eluting peak was consumed.

Experiment 5

Using the protocol described above for resolution, o-fluoro-α-phenylalanine was incubated with cell lysate from Variovorax paradoxus for 67 hours, after which time 99% of the later eluting peak was consumed.

Experiment 6

Using the protocol described above for resolution, 3-amino-4-methylpentanoic acid was incubated with cell lysate from Variovorax paradoxus for 48 hours, after which time 65% of the later eluting peak was consumed.

Experiment 7

Using the protocol described above for resolution, 3-amino-3-cyclohexylpropanoic acid was incubated with cell lysate from Variovorax paradoxus for 2 hours, after which time >99% of the later eluting peak was consumed.

Experiment 8

Using the protocol described above for resolution, 3-amino-3-cyclopropylpropanoic acid was incubated with cell lysate from Variovorax paradoxus for 22 hours, after which time 98% of the later eluting peak was consumed.

Experiment 9

Using the protocol described above for resolution, β-phenylalanine was incubated with cell lysate from Variovorax paradoxus for 5 hours, after which time >99% of the later eluting peak was consumed.

Experiment 10

Using the protocol described above for resolution, p-nitro-α-phenylalanine was incubated with sodium sulfate purified beta-aminotransferase from Variovorax paradoxus. After 30 minutes approximately 98% of the later eluting peak was consumed; after 1 hour it was not detected. After 18 hours there was no detectable change in the concentration of the early eluting stereoisomer.

Experiment 11

Using the protocol described above for resolution, β-phenylalanine was incubated with cell lysate from Alcaligenes eutrophus. After 5 hours approximately 97% of the earlier eluting peak was consumed. After one day the earlier eluting peak was not detectable, and there was no detectable change in the concentration of the later eluting stereoisomer.

Experiment 12

Using the protocol described above for resolution, 3-amino-3-cyclohexyl propanoic acid was incubated with cell lysate from Alcaligenes eutrophus. After 22 hours approximately 37% of the earlier eluting peak was consumed.

Experiment 13

Using the protocol described above for resolution, 3-amino-3-cyclopropylpropanoic acid was incubated with cell lysate from Alcaligenes eutrophus. After 22 hours approximately 42% of the earlier eluting peak was consumed.

Experiment 14

Using the protocol described above for resolution, 3-amino-3-(3-thienyl)propanoic acid was incubated with cell lysate from Alcaligenes eutrophus for 21 hours, after which time 82% of the earlier eluting peak was consumed.

Experiment 15

Using the protocol described above for resolution, 3-amino-3-(2-furyl)propanoic acid was incubated with cell lysate from Alcaligenes eutrophus for 21 hours, after which time 73% of the earlier eluting peak was consumed.

Experiment 16

Using the protocol described above for resolution, 3-amino-3-(2-naphthyl)propanoic acid was incubated with cell lysate from Variovorax paradoxus for 5 hours, after which time 50% of the later eluting peak was consumed.

Experiment 17

Using the protocol described above for resolution, 3-amino-3-(2-benzofuryl))propanoic acid was incubated with cell lysate from Variovorax paradoxus for 5 hours, after which time 98% of the later eluting peak was consumed.

Experiment 18

Using the protocol described above for resolution, 3-amino-3-(3-pyridyl)propanoic acid was incubated with cell lysate from Variovorax paradoxus for 5 hours, after which time >95% of the later eluting peak was consumed.

Experiment 19

Using the protocol described above for resolution, 3-amino-3-(4-phenoxyphenyl)propanoic acid was incubated with cell lysate from Variovorax paradoxus for 5 hours, after which time 58% of the later eluting peak was consumed.

Experiment 20

Using the protocol described above for resolution, 3-aminohexanoic acid was incubated with cell lysate from Variovorax paradoxus for 1 hour, after which time >99% of the later eluting peak was consumed.

Experiment 21

Using the protocol described above for resolution, 2-aminocyclohexanecarboxylic acid was incubated with cell lysate from Variovorax paradoxus for 19 hours, after which time >99% of the later eluting peak(s) was consumed, only the two earlier eluting of each pair of diastereoisomers remaining.

Experiment 22

Using the protocol described above for resolution, 2-aminocyclopentanecarboxylic acid was incubated with cell lysate from Variovorax paradoxus for 24 hours, after which time 45% of the earlier eluting peak of one pair of diastereomers and 20% of the earlier eluting peak of the other pair of diastereomers was consumed.

Experiment 23

Using the protocol described above for synthesis, o-fluoro-phenyl-2-oxopropanoic acid was incubated with sodium sulfate purified β-aminotransferase from Variovorax paradoxus for 3 hours, after which time only later eluting peak was observed, corresponding to approximately 0.02 mg/mL using β-phenylalanine as standard.

Experiment 24

Using the protocol described above for synthesis, 3-Oxohexanoic acid, at a final concentration of approximately 50 mM, was incubated in the presence of approximately 60 mM glutamic acid with cell lysate from Variovorax paradoxus. After 19 hours, only later eluting peak was observed, corresponding to approximately 1 mg/mL 3-aminohexanoic acid, using beta-phenylalanine as standard.

Experiment 25

Using the protocol described above for synthesis, 3-(2-Fluorobenzene)beta-oxopropanoic acid, at a final concentration of approximately 45 mM, was incubated in the presence of approximately 84 mM alanine with cell lysate from Alcaligenes eutrophus. After 47 hours, early eluting to later eluting peak was observed in about 2.5:1 ratio, and corresponding to approximately 0.1 mg/mL 2-fluoro-beta-phenylalanine, using beta-phenylalanine as standard.

Experiment 26

Using the protocol described above for synthesis, 3-Amino-3-cyclopropylpropionic acid, at 0.225 M, was incubated with cell lysate from Variovorax paradoxus and 0.45 M alpha-ketoglutarate, pH 8.1. After 18.2 hours, 99.5% of the early eluting peak was observed. Isolation of the remaining stereoisomer in 6.5 mL of the bioconversion by adsorption to an ion exchange chromatography and elution with ammonium hydroxide gave 80 mg single (early eluting) stereoisomer, with >95% chiral purity.

TABLE 13
Resolution of various substrates with β-aminotransferases

| Experiment | Substrate | Enzyme source | Time | Peak consumption |
|---|---|---|---|---|
| 1 | β-phenylalanine | Variovorax | 23 | 82% |
| | | paradoxus | | |
| 2 | p-methoxy-β-phenylalanine | Variovorax | 23 | 80% |
| | | paradoxus | | |
| 3 | m-nitro-β-phenylalanine | Variovorax | 23 | 95% |
| | | paradoxus | | |
| 4 | p-fluoro-β-phenylalanine | Variovorax | 23 | 82% |
| | | paradoxus | | |
| 5 | o-fluoro-β-phenylalanine | Variovorax | 67 | 99% |
| | | paradoxus | | |
| 6 | 3-amino-4-methylpentanoic | Variovorax | 48 | 65% |
| | | paradoxus | | |
| 7 | 3-amino-3- | Variovorax | 2 | >99% |
| | cyclohexylpropanoic acid | paradoxus | | |
| 8 | 3-amino-3- | Variovorax | 22 | 98% |
| | cyclopropylpropanoic acid | paradoxus | | |
| 9 | β-phenylalanine | Variovorax | 5 | >99% |
| | | paradoxus | | |
| 10 | p-nitro-β-phenylalanine | Variovorax | 0.5 | 98% |
| | | paradoxus | | |
| 11 | β-phenylalanine | Alcaligenes | 5 | 97% |
| | | eutrophus | | |
| 12 | 3-amino-3-cyclohexyl | Alcaligenes | 22 | 37% |
| | propanoic acid | eutrophus | | |
| 13 | 3-amino-3-cyclopropyl | Alcaligenes | 22 | 42% |
| | propanoic acid | eutrophus | | |
| 14 | 3-amino-3-(3- | Alcaligenes | 22 | 82% |
| | thienyl)propanoic acid | eutrophus | | |
| 15 | 3-amino-3-(2-furyl) | Alcaligenes | 21 | 73% |
| | propanoic acid | eutrophus | | |
| 16 | 3-amino-3-(2- | Variovorax | 5 | 50% |
| | naphthyl)propanoic acid | paradoxus | | |
| 17 | 3-amino-3-(2- | Variovorax | 5 | 98% |
| | benzofuryl))propanoic acid | paradoxus | | |
| 18 | 3-amino-3-(3- | Variovorax | 5 | >95% |
| | pyridyl)propanoic acid | paradoxus | | |
| 19 | 3-amino-3-(4- | Variovorax | 5 | 58% |
| | phenoxyphenyl)propanoic | paradoxus | | |
| | acid | | | |
| 20 | 3-aminohexanoic acid | Variovorax | 1 | >99% |
| | | paradoxus | | |
| 21 | 2- | Variovorax | 19 | >99% |
| | aminocyclohexanecarboxylic | paradoxus | | |
| | acid | | | |
| 22 | 2- | Variovorax | 24 | 45% (one pair) |
| | aminocyclopentanecarboxylic | paradoxus | | 20% (other |
| | acid | | | pair) |

### REFERENCES

All references, patents, or applications cited herein are incorporated by reference in their entirety, as if written herein.
Cardillo et al.
Danzin C, Jung M J. Lack of stringent stereospecificity in the inactivation of pyridoxal phosphate-dependent enzymes by suicide-substrates. Prog. Clin. Biol. Res. 1984;144A:377-85 Hayaishi, O., Nishizuka, Y., Tatibana, M., Takeshita, M. and Kuno, S. Enzymatic studies on the metabolism of β-alanine. J. Biol. Chem. 236 (1961) 781-790;
Juaristi, Eusebio, ed. Enantioselective Synthesis of β-Amino Acids, Wiley-VCH, 491 pages, 1997
Kolbert, Christopher P., and Persing, David H Ribosomal DNA sequencing as a tool for identification of bacterial pathogens, Current Opinion in Microbiology, Volume 2, Issue 3, June 1999, Pages 299-305 and Errata, Volume 2, Issue 4, August 1999, Page 452 Nakano et al. J. Biochem 81, 1375-1381, 1977.
Ng and coworkers have reported the PA-catalyzed resolution of β-monosubstituted beta amino acids ( ).
Patel, Jean Baldus, Debra G. B. Leonard, Xai Pan, James M. Musser, Richard E. Berman, and Irving Nachamkin Sequence-Based Identification of Mycobacterium Species Using the MicroSeq 500 16S rDNA Bacterial Identification System J. Clin. Microbiol. 2000 38: 246-251. Pohl, T., Waldmann H, Tetrahedron Lett (1995) 36: 2963-2966; Waldman H. Tetrahedron Lett (1988) 29:1131-1134).
Shin J S, Kim B G. Comparison of the omega-transaminases from different microorganisms and application to production of chiral amines. Biosci Biotechnol Biochem 2001 August; 65(8):1782-8. Microorganisms that are capable of (S)-enantioselective transamination of chiral amines were isolated from soil samples by selective enrichment using (S)-alpha-methyl-benzylamine ((S)-alpha-MBA) as a sole nitrogen source. Among them, Klebsiella pneumoniae JS2F, Bacillus thuringiensis JS64, and Vibrio fluvialis JS17 showed good omega-transaminase (omega-TA) activities and the properties of the omega-TAs were investigated. The induction level of the enzyme was strongly dependent on the nitrogen source for the strains, except for V. fluvialis JS17. All the omega-TAs showed high enantioselectivity (E>50) toward (S)-alpha-MBA and broad amino donor specificities for arylic and aliphatic chiral amines. Besides pyruvate, aldehydes such as propionaldehyde and butyraldehyde showed good amino acceptor reactivities. All the omega-TAs showed substrate inhibition by (S)-alpha-MBA above 200 mm. Moreover, substrate inhibition by pyruvate above 10 mm was observed for omega-TA from V. fluvialis JS17. In the case of product inhibition, acetophenone showed much greater inhibitions than L-alanine for all omega-TAs. Comparison of the enzyme properties indicates that omega-transaminase from V. fluvialis JS17 is the best one for both kinetic resolution and asymmetric synthesis to produce enantiomerically pure chiral amines. Kinetic resolution of sec-butylamine (20 mM) was done under reduced pressure (150 Torr) to selectively remove an inhibitory product (2-butanone) using the enzyme from V. fluvialis JS17. Enantiomeric excess of (R)-sec-butylamine reached 94.7% after 12 h of reaction. Slater, J. H., Lovatt, D., Weightman, A. J., Senior, E., and Bull, A., T., 1979. The Growth of Pseudomonas putida on chlorinated aliphatic acids and it's dehalogenase activity. Journal of General Microbiology 114, 125-136.
Soloshonok et al. (,).
Soloshonok, V. A. Biocatalytic entry to enantiomerically pure β-amino acids. In Enantioselective Synthesis of β-Amino Acids, Edited by Eusebio Juaristi, Wiley-VCH, 1997, 443-464. Stinson, R. A. and Spencer, M. S. β-Alanine aminotransferase(s) from a plant source. Biochem. Biophys. Res. Commun. 34 (1969) 120-127).
Stirling, D. I. 1992. The Use of Aminotransferases for the Production of Chiral Amino Acids and Amines, pp. 209-222. In: Collins, Sheldrake, and Crosby (eds), Chirality in Industry, John Wiley and Sons Ltd., New York. Stirling, D. I. “Enzymic synthesis and resolution of enantiomerically pure compounds” In Chiralty Ind. (1992) 209-22, Wiley, Ed(s). Colins, Andrew N.; Sheldrake, G. N., and Crosby, J.
Stirling, David I., Matcham, George W., and Zeitlin, Andrew I., 1990. Enantiomeric Enrichment and Stereoselective Synthesis of Chiral Amines. U.S. Pat. No. 4,950,606. Stirling, David I., Zeitlin, Andrew I., and Matcham, George W., 1994. Enantiomeric Enrichment and Stereoselective Synthesis of Chiral Amines. U.S. Pat. No. 5,300,437.
Stirling, David I., Zeitlin, Andrew I., Matchum, George W., Rozzell, James D., 1992. Enantiomeric Enrichment and Stereoselective Synthesis of Chiral Amines. U.S. Pat. No. 5,169,780. U.S. Pat. No. 4,518,692 to Rozzell
U.S. Pat. No. 4,826,766 to Rozzell
U.S. Pat. No. 5,316,943 to Kidman
U.S. Pat. No. 6,197,558 to Fotheringham U. S. Pat. No. 4,600,692 to Wood
Watanabe N, Sakabe K, Sakabe N, Higashi T, Sasaki K, Aibara S, Morita Y, Yonaha K, Toyama S, Fukutani H. Crystal structure analysis of omega-amino acid:pyruvate aminotransferase with a newly developed Weissenberg camera and an imaging plate using synchrotron radiation. J Biochem (Tokyo) 1989 January; 105(1):1-3 The three-dimensional structure of omega-amino acid:pyruvate aminotransferase from Pseudomonas sp. F-126, an isologous alpha 4 tetramer containing pyridoxal 5′-phosphate (PLP) as a cofactor, has been determined at 2.0 A resolution. The diffraction data were collected with a newly developed Weissenberg camera with a Fuji Imaging Plate, using synchrotron radiation. The mean figure-of-merit was 0.57. The subunit is rich in secondary structure and comprises two domains. PLP is located in the large domain. The high homology in the secondary structure between this enzyme and aspartate aminotransferase strongly indicates that these two types of enzymes have evolved from a common ancestor. Waters et al. FEMS Micro Lett 34 (1986) 279-282.
West T P. Role of cytosine deaminase and beta-alanine-pyruvate transaminase in pyrimidine base catabolism by Burkholderia cepacia. Antonie Van Leeuwenhoek 2000 January; 77(1):1-5 A determination of the possible role of the salvage enzyme cytosine deaminase or beta-alanine-pyruvate transaminase in the catabolism of the pyrimidine bases uracil and thymine by the opportunistic pathogen Burkholderia cepacia ATCC 25416 was undertaken. It was of interest to learn whether these enzymes were influenced by cell growth on pyrimidine bases and their respective catabolic products to the same degree as the pyrimidine reductive catabolic enzymes were. It was found that cytosine deaminase activity was influenced very little by cell growth on the pyrimidines tested. Using glucose as the carbon source, only B. cepacia growth on 5-methylcytosine as a nitrogen source increased deaminase activity by about three-fold relative to (NH4)2SO4-grown cells. In contrast, the activity of beta-alanine-pyruvate transaminase was observed to be at least double in glucose-grown ATCC 25416 cells when pyrimidine bases and catabolic products served as nitrogen sources instead of (NH4)₂SO4. Transaminase activity in the B. cepacia glucose-grown cells was maximal after the strain was grown on either uracil or 5-methylcytosine as a nitrogen source compared to (NH4)₂SO4-grown cells. A possible role for beta-alanine-pyruvate transaminase in pyrimidine base catabolism by B. cepacia would seem to be suggested from the similarity in how its enzyme activity responded to cell growth on pyrimidine bases and catabolic products when compared to the response of the three reductive catabolic enzymes. Yonaha and coworkers Agric. Biol. Chem. 41(9): 1701-1706, 1977).
Yonaha and coworkers Agric. Biol. Chem. 42(12): 2363-2367, 1978;
Yonaha K, Nishie M, Aibara S. The primary structure of omega-amino acid:pyruvate aminotransferase. J Biol Chem 1992 Jun. 25;267(18):12506-10 The complete amino acid sequence of bacterial omega-amino acid:pyruvate aminotransferase (omega-APT) was determined from its primary structure. The enzyme protein was fragmented by CNBr cleavage, trypsin, and Staphylococcus aureus V8 digestions. The peptides were purified and sequenced by Edman degradation. omega-ATP is composed of four identical subunits of 449 amino acids each. The calculated molecular weight of the enzyme subunit is 48,738 and that of the enzyme tetramer is 194,952. No disulfide bonds or bound sugar molecules were found in the enzyme structure, although 6 cysteine residues were determined per enzyme subunit. Sequence homologies were found between an omega-aminotransferase, i.e. mammalian and yeast ornithine delta-aminotransferases, fungal gamma-aminobutyrate aminotransferase and 7,8-diaminoperalgonate aminotransferase, and 2,2-dialkylglycine decarboxylase. The enzyme structure is not homologous to those of aspartate aminotransferases (AspATs) including the enzymes of Escherichia coli and Sufolobus salfactaricus, though significant homology in the three-dimensional structures around the cofactor binding site has been found between omega-APT and AspATs (Watanabe, N., Sakabe, K., Sakabe, N., Higashi, T., Sasaki, K., Aibara, S., Morita, Y., Yonaha, K., Toyama, S., and Fukutani, H. (1989) J. Biochem. 105, 1-3). Yonaha K, Toyama S, Kagamiyama H. Omega-amino acid: pyruvate aminotransferase: subunit structure, spectrometric properties and amino acid sequence around pyridoxyl lysine. Prog Clin Biol Res 1984;144B: 329-38
Yonaha K, Toyama S, Kagamiyama H. Properties of the bound coenzyme and subunit structure of omega-amino acid:pyruvate aminotransferase. J Biol Chem 1983 Feb. 25;258(4):2260-5.

1. A process for the stereoselective synthesis of a β-amino acid, or a salt thereof, the process comprising contacting an amino donor and an amino acceptor in the presence of a β-amino acid transaminase to form a β-amino acid enantiomer, or a salt thereof, from the amino acceptor.

2. The process of claim 1 wherein the amino acceptor is a β-keto acid.

3. The process of claim 1 wherein the amino donor is an α-amino acid.

4. The process of claim 1, wherein the molar ratio of the D-β-amino acid or L-β-amino acid formed to the respective L-β-amino acid or D-β-amino acid formed is greater than 1:1.

5. The process of claim 4, wherein the molar ratio is greater than 3:1.

6. The process of claim 5, wherein the molar ratio is greater than 10:1.

7. The process of claim 1, further comprising recovering the β-amino acid.

8. The process of claim 1, wherein said contacting is carried out in the presence of whole cells of a microorganism which comprises the β-transaminase.

9. The process of claim 1, wherein said contacting is carried out in the presence of permeabilized cells of a microorganism which comprises the β-transaminase.

10. The process of claim 1, wherein said contacting is carried out in the presence of a cell-free preparation of the β-transaminase.

11. The process of claim 1 wherein the β-transaminase is immobilized on a support.

12. The process of claim 1 wherein the contacting is carried out in aqueous conditions.

13. The process of claim 1 wherein the contacting is carried out in the presence of an organic cosolvent.

14. The process of claim 13 wherein the organic cosolvent is selected from the group consisting of alcohols, ketones, ethers, esters, nitriles, and hydrocarbons.

15. The process of claim 13 wherein the organic cosolvent chosen from the group consisting of methanol, ethanol, propanol, isopropanol, acetone, diethyl ether, ethyl acetate, tetrahydrofuran, dimethylformamide, acetonitrile, methyl t-butyl ether, di-octyl phthalate, toluene, dialkyl ether, and diphenyl ether.

16. The process of claim 15 wherein the organic cosolvent is present in an amount between 0% and 100% (v/v).

17. The process of claim 16 wherein the organic cosolvent is present in an amount between 0% and about 30% (v/v).

18. The process of claim 17 wherein the organic cosolvent is present in an amount of about 5% (v/v).

19. The process of claim 1 where the organic cosolvent is water miscible.

20. The process of claim 1 where the organic cosolvent is water immiscible.

21. The process of claim 1, further comprising reacting the corresponding keto form of the amino donor, produced by contacting an amino donor and an amino acceptor in the presence of a β-amino acid transaminase, under conditions appropriate to produce a compound that does not react with the β-transaminase.

22. The process of claim 21, wherein the keto form of the amino donor is an alpha keto acid.

23. The process of claim 22, wherein the amino donor is glutamate, and the keto form of the amino donor is α-keto glutarate.

24. The process of claim 23, wherein the amino donor is glutamate, the keto form of the amino donor is a-keto glutarate, and the reacting is carried out in the presence of asp-oxaloacetate transaminase and oxaloacetate decarboxylase.

25. The process of claim 21, wherein the keto form of the amino donor is pyruvic acid.

26. The process of claim 25, wherein the amino donor is L-alanine, the keto form of the amino donor is pyruvic acid, and the reacting is carried out in the presence of pyruvate decarboxylase.

27. The process of claim 1 wherein the β-amino acid enantiomer is a D-β-amino acid enantiomer.

28. The process of claim 27, wherein the β-amino acid enantiomer is a D-β-amino acid and the transaminase is a stereoselective D-β-transaminase.

29. The process of claim 28, wherein said transaminase is derived from a microorganism selected from the genera consisting of Variovorax, Nocardia, Comamonas, Rhodococcus, and Pseudomonas.

30. The process of claim 29, wherein said transaminase is derived from a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

31. The process of claim 28, wherein said transaminase is substantially identical to a stereoselective D-β-transaminase produced by a microorganism selected from the genera consisting of Variovorax, Nocardia, Comamonas, Rhodococcus, and Pseudomonas.

32. The process of claim 31, wherein said transaminase is substantially identical to a stereoselective D-β-transaminase produced by a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

33. The process of claim 28, wherein said transaminase is at least 80% identical to the amino acid sequence of a stereoselective D-β-transaminase produced by a microorganism selected from the genera consisting of Variovorax, Nocardia, Comamonas, Rhodococcus, and Pseudomonas.

34. The process of claim 33, wherein said transaminase is at least 80% identical to the amino acid sequence of a stereoselective D-β-transaminase produced by a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

35. The process of claim 1 wherein the β-amino acid enantiomer is the L-β-amino acid enantiomer.

36. The process of claim 35, wherein an L-β-amino acid is synthesized in the presence of a stereoselective L-β-transaminase.

37. The process of claim 36, wherein said transaminase is derived from a microorganism of the genus Alcaligenes.

38. The process of claim 37, wherein said transaminase is produced by Alcaligenes eutrophus.

39. The process of claim 36, wherein said transaminase is substantially identical to a stereoselective L-β-transaminase produced by a microorganism of the genus Alcaligenes.

40. The process of claim 39, wherein said transaminase is substantially identical to a stereoselective L-β-transaminase produced by Alcaligenes eutrophus.

41. The process of claim 36, wherein said transaminase is at least 80% identical to the amino acid sequence of a stereoselective L-β-transaminase produced by a microorganism of the genus Alcaligenes.

42. The process of claim 41, wherein said transaminase is at least 80% identical to the amino acid sequence of a stereoselective L-β-transaminase produced by Alcaligenes eutrophus.

43. The process of claim 1 wherein the β-amino acid enantiomer is the D-β-amino acid enantiomer synthesized in the presence of a stereoselective D-α-transaminase, wherein said transaminase is derived from a microorganism having at least 97% identity over 1500 nucleotides with the 16S rRNA sequence set forth in SEQ ID NO:1.

44. The process of claim 1 wherein the β-amino acid enantiomer is the D-β-amino acid enantiomer synthesized in the presence of a stereoselective D-β-transaminase, wherein said transaminase is derived from a microorganism having at least 97% identity over 1500 nucleotides with the 16S rRNA sequence set forth in SEQ ID NO:2.

45. The process of claim 1, wherein the β-amino acid is a compound of Formula I 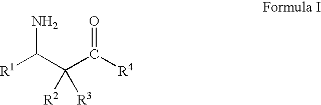
and the amino acceptor is a compound of Formula II 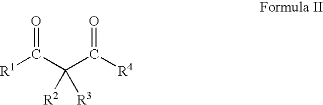
wherein R¹, R², and R³ are independently selected from the group consisting of hydrogen, C₁₋₈alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl;
wherein R¹, R², and R³ are not all H; and
R⁴ comprises hydroxy, O⁻, and —OM; wherein M is a cation.

46. The process of claim 45 wherein M is selected from the group consisting of alkali metal cations and NH₄ ⁺.

47. The process of claim 46 wherein M is selected from the group consisting of Na⁺, K⁺, and NH₄ ⁺.

48. The process of claim 45 wherein R¹, R², and R³ are selected from the group consisting of hydrogen, C₁₋₈ alkyl, C₂₋₈ alkenyl, C₂₋₈ alkynyl, C₆₋₁₂ aryl, and C₆₋₁₂ aryl-C₁₋₈ alkyl, radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl radicals.

49. The process of claim 48 wherein R¹, R², and R³ are independently selected from the group consisting of hydrogen, C₆₋₁₂ aryl, and C₆₋₁₂ aryl-C₁₋₈ alkyl, radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl radicals.

50. The process of claim 48 wherein R¹, R², and R³ are selected from the group consisting of hydrogen, C₁₋₈ alkyl, C₂₋₈ alkenyl, and C₂₋₈ alkynyl, radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl radicals.

51. The process of claim 48, wherein R² or R³, but not both, is OH.

52. The process of claim 48, wherein R² or R³, but not both, is H.

53. The process of claim 48, wherein R² and R³ are both H.

54. The process of claim 53, wherein R¹ is selected from the group consisting of C₆₋₁₂ aryl and C₆₋₁₂ aryl-C₁₋₈ alkyl radicals, which are optionally substituted with halogen, nitro, thio, lower alkoxy, and lower alkyl radicals.

55. The process of claim 54, wherein R¹ is phenyl.

56. A process for the stereoselective synthesis of a β-amino acid, its salt, the process comprising contacting an amino donor and an amino acceptor in the presence of a β-amino acid transaminase to stereoselectively form a amino acid enantiomer, or a salt thereof, from the amino acceptor;
wherein the β-amino acid, or a salt thereof, is a compound of Formula III 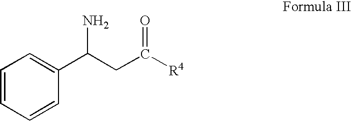
and the amino acceptor is a compound of Formula IV: 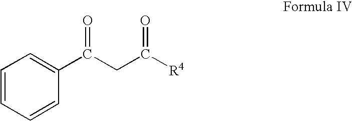
wherein R⁴ comprises hydroxy, O⁻, and —OM; wherein M is a cation.

57. The process of claim 56, wherein the β-amino acid is selected from the group consisting of D-β-phenylalanine and L-β-phenylalanine.

58. The process of claim 56, wherein the amino acceptor is selected from the group consisting of a β-keto acid and a compound converted to β-keto acid in situ.

59. The process of claim 56, wherein the amino donor is selected from the group consisting of:
D-glutamic acid, L-glutamic acid, D,L-glutamic acid,
D-aspartic acid, L-aspartic acid, D,L-aspartic acid,
D-alanine, L-alanine, and D,L-alanine,
3-aminoadipic acid, and
2-aminoadipic acid.

60. The process of claim 59, wherein the amino donor is selected from the group consisting of:
D-glutamic acid, L-glutamic acid, D,L-glutamic acid,
D-aspartic acid, L-aspartic acid, and D,L-aspartic acid.

61. A process for enantiomerically enriching a mixture comprising a D-β-amino acid enantiomer and its corresponding L-β-amino acid enantiomer, the process comprising contacting the L-β-amino acid enantiomer with an amino acceptor in the presence of a stereoselective L-β-transaminase to convert at least a portion of the L-β-amino acid enantiomer to the corresponding β-keto acid thereby increasing the molar ratio of the D-β-amino acid enantiomer to the L-β-amino acid enantiomer in the enriched mixture.

62. The process of claim 61, wherein the molar ratio of D-β-amino acid enantiomer to L-β-amino acid enantiomer in the enriched mixture is greater than 1:1.

63. The process of claim 62, wherein the molar ratio of D-β-amino acid enantiomer to L-β-amino acid enantiomer in the enriched mixture is greater than 3:1.

64. The process of claim 63, wherein the molar ratio of D-β-amino acid enantiomer to L-β-amino acid enantiomer in the enriched mixture is greater than 10:1.

65. A process for enantiomerically enriching a mixture comprising an L-β-amino acid enantiomer and its corresponding D-β-amino acid enantiomer, the process comprising contacting the D-β-amino acid enantiomer with an amino acceptor in the presence of a stereoselective D-β-transaminase to convert at least a portion of the D-β-amino acid enantiomer to the corresponding β-keto acid thereby increasing the molar ratio of the L-β-amino acid enantiomer to the D-β-amino acid enantiomer in the enriched mixture.

66. The process of claim 65, wherein the molar ratio of L-β-amino acid enantiomer to D-β-amino acid enantiomer in the enriched mixture is greater than 1:1.

67. The process of claim 66, wherein the molar ratio of L-β-amino acid enantiomer to D-β-amino acid enantiomer in the enriched mixture is greater than 3:1.

68. The process of claim 67, wherein the molar ratio of L-β-amino acid enantiomer to D-β-amino acid enantiomer in the enriched mixture is greater than 10:1.

69. A method for preparing an enantiomerically enriched β-amino acid, or a salt thereof, which comprises contacting
(i) a racemic β-amino acid, or salt thereof, having the structure of Formula I: 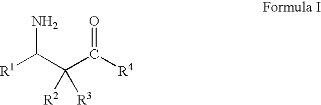
wherein R¹, R², and R³ are independently selected from the group consisting of hydrogen, C₁₋₈ alkyl, C₂₋₈ alkenyl, C₂₋₈ alkynyl, C₃₋₁₂ cycloalkyl, C₆₋₁₂ aryl, C₃₋₁₂ heterocyclyl, C₆₋₁₂ aryl-C₁₋₈ alkyl, and C₃₋₁₂ heterocyclyl-C₁₋₈ alkyl radicals;
wherein all of said radicals are optionally substituted with hydroxyl, lower alkoxy, lower alkyl, halogen, nitro, carboxyl, trifluoromethyl, amino, acyloxy, phenyl, benzyl, naphthyl, quinoline, isoquinoline, which are optionally substituted with halogen, nitro, thio, lower alkoxy, and lower alkyl;
wherein R¹, R², and R³ are not all H; and
R⁴ comprises hydroxy, O⁻, and —OM; wherein M is a cation;
(ii) an amino acceptor, and
(iii) a stereospecific β-amino acid transaminase;
under conditions appropriate to convert one enantiomer of the racemic β-amino acid to its corresponding β-keto acid derivative, whereby the opposite enantiomer of the β-amino acid is retained in substantially enantiomerically enriched form, and separating the β-keto acid derivative from the retained β-amino acid.

70. A purified stereoselective D-β-transaminase derived from a microorganism selected from the group consisting of Variovorax, Nocardia, Comamonas, Rhodococcus, and Pseudomonas.

71. A purified stereoselective D-β-transaminase of claim 70 derived from a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

72. A purified stereoselective D-β-transaminase of claim 71 wherein the sequence of the 16S rDNA of said microorganism has at least 97% identity over 1500 nucleotides with the sequence of the 16S rDNA set forth in SEQ ID NO:1.

73. A purified stereoselective D-β-transaminase of claim 71 derived from Variovorax paradoxus.

74. A purified stereoselective D-β-transaminase of claim 73 derived from Variovorax paradoxus, wherein the sequence of the 16S rDNA of said microorganism comprises SEQ ID NO: 1.

75. A purified stereoselective D-β-transaminase of claim 73 derived from Variovorax paradoxus, wherein the sequence of the 16S rDNA of said microorganism consists of SEQ ID NO: 1.

76. A purified stereoselective D-β-transaminase of claim 71 wherein the sequence of the 16S rDNA of said microorganism has at least 97% identity over 1500 nucleotides with the sequence of the 16S rDNA set forth in SEQ ID NO:2.

77. A purified stereoselective D-β-transaminase of claim 71 derived from Rhodococcus opacus.

78. A purified stereoselective D-β-transaminase of claim 77 derived from Rhodococcus opacus, wherein the sequence of the 16S rDNA of said microorganism comprises SEQ ID NO: 2.

79. A purified stereoselective D-β-transaminase of claim 77 derived from Rhodococcus opacus, wherein the sequence of the 16S rDNA of said microorganism consists of SEQ ID NO: 2.

80. A purified stereoselective L-β-transaminase derived from a microorganism of the genus Alcaligenes.

81. The purified stereoselective L-β-transaminase of claim 80, derived from Alcaligenes eutrophus.

82. A process for purifying a stereospecific β-transaminase from a cell homogenate comprising the stereospecific β-transaminase, the process comprising contacting the cell homogenate with a precipitating agent to yield a precipitate comprising the stereospecific β-transaminase.

83. The process of claim 82, wherein the precipitating agent is ammonium sulfate.

84. The process of claim 82, wherein the precipitate is further purified by chromatography.

85. The process of claim 82, wherein the precipitate is further purified by hydrophobic interaction chromatography.

86. The process of claim 85, wherein the hydrophobic interaction chromatography is performed with a butyl sepharose. FF resin.

87. The process of claim 82, wherein the precipitate is further purified by size exclusion chromatography.

88. The process of claim 87, wherein the size exclusion chromatography is performed with a TSK G300 SW resin.

89. The process of claim 82, wherein the precipitate is further purified by hydrophobic interaction chromatography and size exclusion chromatography.

90. The process of claim 89, wherein the hydrophobic interaction chromatography is performed with a butyl sepharose FF resin and the size exclusion chromatography is carried out with a TSK G300 SW resin.

91. A stereoselective D-β-transaminase produced by the process of claim 82 wherein the cell homogenate is obtained from a microorganism selected from the group consisting of Variovorax, Nocardia, Comamonas, Rhodococcus, and Pseudomonas.

92. A stereoselective D-β-transaminase produced by the process of claim 91 wherein the cell homogenate is obtained from a microorganism selected from the group consisting of Variovorax paradoxus, Variovorax paradoxus GC subgroup A, Nocardia asteroides, Comamonas terrigena, Pseudomonas mendocina, Comamonas acidovorans, and Rhodococcus opacus.

93. The stereoselective D-β-transaminase produced by the process of claim 92 wherein the cell homogenate is obtained from Variovorax paradoxus.

94. The stereoselective D-β-transaminase of claim 93 produced by Variovorax paradoxus, wherein the 16S rDNA of said microorganism comprises SEQ ID NO: 1.

95. The stereoselective D-β-transaminase of claim 93 produced by Variovorax paradoxus, wherein the 16S rDNA of said microorganism consists of SEQ ID NO: 1.

96. The stereoselective D-β-transaminase produced by the process of claim 92 wherein the cell homogenate is obtained from Rhodococcus opacus.

97. The stereoselective D-α-transaminase of claim 96 produced by Rhodococcus opacus, wherein the 16S rDNA of said microorganism comprises SEQ ID NO: 2.

98. The stereoselective D-β-transaminase of claim 96 produced by Rhodococcus opacus, wherein the 16S rDNA of said microorganism consists of SEQ ID NO: 2.

99. A stereoselective L-β-transaminase produced by the process of claim 82, wherein the cell homogenate is obtained from a microorganism of the genus Alcaligenes.

100. A stereoselective L-β-transaminase produced by the process of claim 99, wherein the microorganism is Alcaligenes eutrophus.

101. A β-transaminase produced by the process of claim 82 and having a subunit molecular weight between 45 and 55 kDa.

102. A process for purifying a stereospecific β-transaminase from a composition comprising a stereospecific β-transaminase, the process comprising the steps of:
(a) adsorbing the stereospecific β-transaminase onto an hydrophobic interaction material, and
(b) eluting the stereospecific β-transaminase from the hydrophobic interaction material using an elution buffer.

103. A process for purifying a stereospecific β-transaminase from a composition comprising a stereospecific β-transaminase, the process comprising the steps of:
(a) adsorbing the stereospecific β-transaminase onto a size exclusion material, and
(b) eluting the stereospecific β-transaminase from the size exclusion material using an elution buffer.

104. A process for enriching a population of microorganisms for one or more microorganisms expressing a β-transaminase, the process comprising growing the population of microorganisms in a culture medium comprising a β-amino acid, or a salt thereof, as a selective nitrogen source.

105. The process of claim 104 wherein the β-transaminase is a stereospecific β-transaminase.

106. The process of claim 105 wherein the stereospecific β-transaminase is a D-β-transaminase.

107. The process of claim 105 wherein the stereospecific β-transaminase is an L-β-transaminase.

108. The process of claim 104, wherein the β-amino acid is selected from the group consisting of a D-β-amino acid, an L-β-amino acid, or a mixture thereof.

109. The process of claim 108 wherein the β-amino acid is selected from the group consisting of a D-β-phenylalanine, and L-β-phenylalanine, or a mixture thereof.

110. The process of claim 104, wherein the culture medium comprises inorganic salts, a carbon source, and a nitrogen source, wherein said β-amino acid, or a salt thereof, is the nitrogen source used for selective enrichment.

111. The process of claim 104, wherein culture medium comprises inorganic salts, a carbon source, and a nitrogen source, wherein said β-amino acid, or a salt thereof, is the nitrogen source and the carbon source.

112. The process of claim 104, wherein the population of microorganisms are collected from soil.

113. A purified culture comprising Variovorax paradoxus, wherein the sequence of the 16S rDNA of said Variovorax paradoxus comprises SEQ ID NO: 1.

114. A purified culture comprising Rhodococcus opacus, wherein the sequence of the 16S rDNA of said Rhodococcus opacus comprises SEQ ID NO: 2.

115. A purified nucleic acid comprising the 16S rDNA sequence set forth in SEQ ID NO: 1, or its complement.

116. A purified nucleic acid comprising the RNA equivalent of claim 115.

117. A nucleic acid that specifically hybridizes under high stringency conditions to a nucleic acid of claim 115.

118. A nucleic acid fragment comprising a fragment of the 16S rDNA sequence set forth in SEQ ID NO: 1, or its complement, having a length of 300 to 1500 nucleotides.

119. A purified nucleic acid comprising the RNA equivalent of claim 118.

120. A nucleic acid that specifically hybridizes under high stringency conditions to a nucleic acid of claim 118.

121. A purified nucleic acid comprising the 16S rDNA sequence set forth in SEQ ID NO: 2, or its complement.

122. A purified nucleic acid comprising the RNA equivalent of claim 121.

123. A nucleic acid that specifically hybridizes under high stringency conditions to a nucleic acid of claim 121.

124. A nucleic acid fragment comprising a fragment of the 16S rDNA sequence set forth in SEQ ID NO: 2, or its complement, having a length of 300 to 1500 nucleotides.

125. A purified nucleic acid comprising the RNA equivalent of claim 124.

126. A nucleic acid that specifically hybridizes under high stringency conditions to a nucleic acid of claim 124.

127. A method of detecting a nucleic acid comprising:
(a) incubating a first nucleic acid with a second nucleic acid obtained or derived from a cell, wherein the first nucleic acid comprises at least 50 nucleotides of SEQ NO: 1, its RNA equivalent, or their full complements, or a nucleic acid with at least 97% identity to about 100 nucleotides of SEQ NO:1, its RNA equivalent, or their full complements,
(b) permitting hybridization between said first nucleic acid and said second nucleic acid; and
(c) detecting the presence of hybridization to said first nucleic acid.

128. The method of claim 127 wherein said first nucleic acid comprises at least 100 nucleotides of SEQ NO: 1, its RNA equivalent, or their full complements.

129. The method of claim 127 wherein said first nucleic acid comprises at least 150 nucleotides of SEQ NO: 1, its RNA equivalent, or their full complements.

130. The method of claim 127 wherein said first nucleic acid comprises at least 200 nucleotides of SEQ NO: 1, its RNA equivalent, or their full complements.

131. The method of claim 127 wherein said first nucleic acid comprises at least 250 nucleotides of SEQ NO: 1, its RNA equivalent, or their full complements.

132. The method of claim 127 wherein said first nucleic acid comprises at least 300 nucleotides of SEQ NO: 1, its RNA equivalent, or their full complements.

133. A method of detecting a nucleic acid comprising:
(A) incubating a first nucleic acid with a second nucleic acid obtained or derived from a cell, wherein the first nucleic acid comprises at least 50 nucleotides of SEQ NO: 2, its RNA equivalent, or their full complements, or a nucleic acid with at least 97% identity to about 100 nucleotides of SEQ NO: 2, its RNA equivalent, or their full complements;
(B) permitting hybridization between said first nucleic acid and said second nucleic acid; and
(C) detecting the presence of hybridization to said first nucleic acid.

134. The method of claim 133 wherein said first nucleic acid comprises at least 100 nucleotides of SEQ NO: 2, its RNA equivalent, or their full complements.

135. The method of claim 133 wherein said first nucleic acid comprises at least 150 nucleotides of SEQ NO: 2, its RNA equivalent, or their full complements.

136. The method of claim 133 wherein said first nucleic acid comprises at least 200 nucleotides of SEQ NO: 2, its RNA equivalent, or their full complements.

137. The method of claim 133 wherein said first nucleic acid comprises at least 250 nucleotides of SEQ NO: 2, its RNA equivalent, or their full complements.

138. The method of claim 133 wherein said first nucleic acid comprises at least 300 nucleotides of SEQ NO: 2, its RNA equivalent, or their full complements.
